(19) United States
(12) Patent Application Publication
(10) Pub. No.: US 2022/0395608 A1
(43) Pub. Date: Dec. 15, 2022

(54) WATER SOLUBLE BIOADHESIVES

(71) Applicant: THE GOVERNING COUNCIL OF THE UNIVERSITY OF TORONTO, Toronto (CA)

(72) Inventors: Paul SANTERRE, Toronto (CA); Eli SONE, Toronto (CA); Michael FLOROS, Toronto (CA)

(21) Appl. No.: 17/787,531

(22) PCT Filed: Dec. 21, 2020
(86) PCT No.: PCT/CA2020/051784

§ 371 Date: Jun. 20, 2022

(30) Foreign Application Priority Data
Dec. 20, 2019 (GB) 1919026.3

(51) Int. Cl. A61L 24/04; A61L 24/00

(52) U.S. Cl. CPC A61L 24/046; A61L 24/0015; A61L 24/0042

(57) ABSTRACT

A water soluble biocompatible adhesive is provided comprising one or more compounds of structure 1 wherein B is an oligomer derived from a polyether or polyalkylene glycol, with a MW<5,000 g/mol, Linker L is a urethane, urea bond, or amide bond; Linker L′ is a urethane or urea bond, A is a chain extender of Mw≤3000 g/mol comprising substituted or unsubstituted alkyl, cycloalkyl and/or aromatic groups, W is a terminal adhesive benzene-1,2-diol derivative or a terminal adhesive benzene-1,2,3-diol derivative, m is 0 or 1; and n is 0, 1, 2, 3 or 4. The compound(s) have a Tg lower than 25° C. The biocompatible adhesive is suitable for use without solvent.

This application claims priority to United Kingdom patent application no. 1919026.3 filed Dec. 20, 2019.

### FIELD OF THE INVENTION

The invention relates to synthetic water soluble biomedical adhesive compounds.

### BACKGROUND

Most commercially available adhesives suffer major compromises in bond strength, curing time and/or biocompatibility, rendering them inappropriate for many biomedical applications. The vast majority of conventional adhesives are not biodegradable or resorbable and lack suitable mechanical properties to be used as tissue adhesives or sealants.

Adhesives which have been approved for clinical use include fibrin, cyanoacrylate, gelatin-resorcin formaldehyde/glutaraldehyde, and PEG-based glues. These adhesive systems all are limited by low bonding under certain conditions and/or poor cytocompatibility [1-8].

Known PEG-based adhesives, which are extensively used, are relatively non-degradable and are associated with adverse swelling issues which can cause nerve and organ damage [7].

Catechol-containing polymeric adhesives inspired by aquatic, marine mussels are of interest due to the natural ability of mussels to bind to a multitude of surfaces under heavily biofouled conditions. Mussel adhesion is reversible and capable of withstanding fluctuations in temperature and ionic strength [9-11]. The four main components of the mussel adhesive are: 1) acid mucopolysaccharides which act as primers; 2) adhesive proteins consisting mainly of polyphenolic proteins rich in the catechol 3,4-dihydroxyphenylalanine (L-DOPA) and lysine; 3) fibrous proteins that act as an attachment thread between mussel and substrate; and 4) polyphenoloxidase to promote intermolecular cross-linking [12, 13].

Branched PEG and grafted catechol-containing polymer derivatives have demonstrated adhesion to biological tissues, but the requirement for solvents and long curing durations renders them unsuitable for most clinical applications [14-17]. In general, these systems are composed of non-degradable polymers and require the use of organic solvents which may be toxic and impede curing and bonding of the adhesive. Organic solvent removal is further required for the adhesives to function optimally and is generally accomplished by evaporation or other means of removal. The lengthy duration of the solvent removal process (on average lasting up to multiple days) is an important consideration when designing biocompatible adhesives [5, 18].

U.S. Pat. No. 8,673,286 discloses poly(alkylene oxide) adhesives using branched (10-20 kDa-PEG)₄ backbones, functionalized with DOPA4, DOPA-Boc, DOPA3-Lys₂, DOHA or DOPA with ester or urethane linkages. These adhesives must be solubilized in phosphate buffer solution (PBS) and mixed in situ by hand in the presence of a water-solubilized oxidant (NalO₄) prior to use.

PCT Pat. Appl. Publ. No. WO 2017/044896 discloses high molecular weight adhesives made of linear and branched structures made from PEG or PEGylated polyols, functionalized with DOPA using isocyanates. The disclosed formulations require the use of large amounts of filler material (>50%), such as dicalcium phosphate. The exemplified adhesives further include long PEG segments (MW>6000) which are non-biodegradable as well as hexamethyl diisocyanate which can produce toxic alkyl diamine biproducts when degraded.

U.S. Pat. No. 8,916,652 discloses branched structures made from PEG-based polyol oligomers. Structures described in the patent are functionalized internally with DOPA, DOPA-Lys, or DOHA using amide/urea linkages. Several disclosed formulations further contain ester or urethane linkages within the branches. All of these disclosed formulations, however, require the use of organic or aqueous based solvent for application; in one example the adhesives are prepared for use by separately dissolving the polymer and NalO₄ as a crosslinking agent in PBS and mixing at a ratio of 1:1 before being spread onto the surface using a spray apparatus.

US Pat. Appl. Publ. No. 2010/0137903 discloses substrates (e.g. films, meshes, etc.) which are treated with high molecular weight bioadhesives based on branched PEG oligomers for use in hernia repairs. Application of these adhesives onto substrates requires a curing procedure in which a first oxidant is added to the adherend substrate and then a weight is placed over the adhesive interface and held stationary for at least 1 hour. In some cases, a second application of oxidant is employed. A prolonged application period as described in this application is not practical for many applications.

U.S. Pat. No. 8,754,285 B2 discloses the synthesis of thin films designed to adhere to a wide assortment of surfaces. The polymer adhesives consist of PCL, PEG and DOPA groups linked through ester and urethane linkages. Although these catechol-containing hydrogels have demonstrated pH-triggered curing and have potential for use as surgical adhesives [20], their complex preparation (constant stirring and careful pH control) limits their practical clinical applications. Furthermore the adhesives disclosed in the patent use toxic organic solvents and periodate oxidants. Furthermore, the complexity of this application process is significant, and much more difficult than the current standard of care.

All of the above adhesives suffer from similar disadvantages selected from complex, application strategies involving organic or aqueous based solvents and the combination of a oxidant in many cases, both of which often has challenges with respect to toxicity. Organic solvents are very often required for application, and spreading and surface penetration strategies may be required. These limitations highlight the need for alternate safe bioadhesives that are easy to process and apply.

### BRIEF SUMMARY

The present disclosure provides:

In a first embodiment, an adhesive comprising:

a compound of structure 1

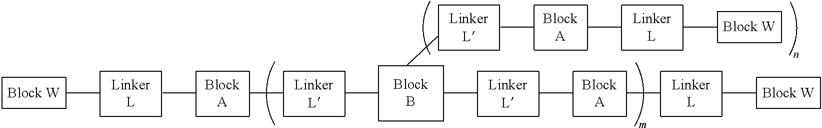

### Structure I

wherein
the compound has a Tg lower than 25° C.;
wherein the compound is water soluble;
B is a branched or unbranched oligomer derived from a polysaccharide, polyamide,
polyether, or polyalkylene glycol, with a MW<5,000 g/mol;
Linker L is an urethane, urea bond, or amide bond;
Linker L′ is an urethane or urea bond;
A is a chain extender of Mw≤3000 g/mol comprising substituted or unsubstituted alkyl, cycloalkyl and/or aromatic groups;
W is a terminal adhesive benzene-1,2-diol derivative or a terminal adhesive benzene-1,2,3-triol derivative;
m is 0 or 1; and
n is 0, 1, 2, 3 or 4;
or
a cross-linked polymer produced by cross-linking compounds of structure 1.
2. The adhesive of embodiment 1, wherein m is 1 and the adhesive comprises a compound of structure 2

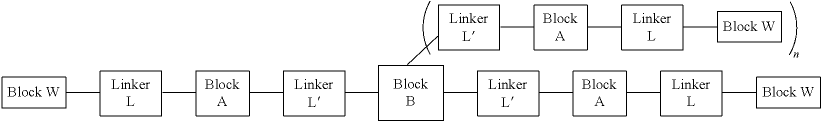

### Structure 2

or
a cross-linked polymer produced by cross-linking compounds of structure 2.
3. The adhesive of embodiment 2 wherein n is 0 and the adhesive comprises a compound of structure 3

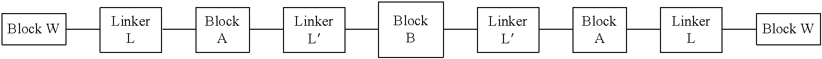

### Structure 3

or
a cross-linked polymer produced by cross-linking compounds of structure 3.
4. The adhesive of embodiment 2 wherein n is 1 and the adhesive comprises a compound of structure 4

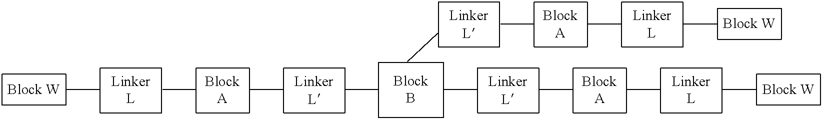

### Structure 4

or
a cross-linked polymer produced by cross-linking compounds of structure 4.
5. The adhesive of embodiment 2 wherein n is 2 and the adhesive comprises a compound of structure 5

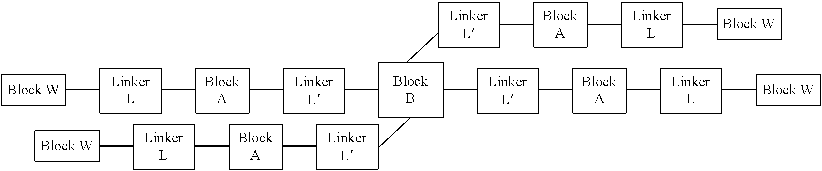

### Structure 5

or
a cross-linked polymer produced by cross-linking compounds of structure 5.
6. The adhesive of embodiment 2 wherein n is 3 and the adhesive comprises a compound of structure 6

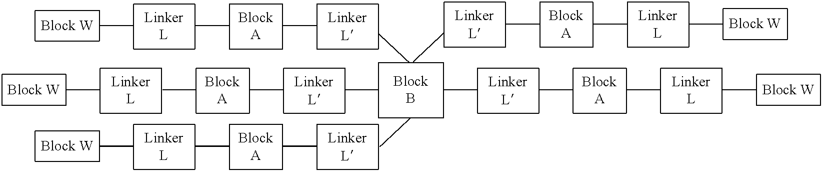

### Structure 6

or
a cross-linked polymer produced by cross-linking compounds of structure 6.
7. The adhesive of embodiment 2 wherein n is 4 and the adhesive comprises a compound of structure 7

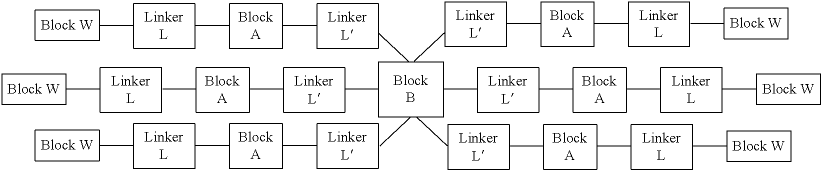

### Structure 7

or
a cross-linked polymer produced by cross-linking compounds of structure 7.
8. The adhesive of any preceding embodiments wherein the compound has a Tm equal to or greater than 37° C.
9. The adhesive of any preceding embodiments wherein B has a Mw<3,000 g/mol, preferably between 500 and 1500 g/mol.
10. The adhesive of any preceding embodiments comprising a compound of structure 1 and crosslinking agent or a curing agent in an amount between about 0 and 5%.
11. The adhesive of any preceding embodiments wherein B is an oligomer derived from a polyether or a polyalkylene glycol, preferably polypropylene glycol or polyethylene glycol.
12. The adhesive of any preceding embodiments wherein A is substituted with a C1-C6 carboxylate group.
13. The adhesive of any preceding embodiments wherein A has a MW<200 g/mol.
14. The adhesive of any preceding embodiments wherein W is a 4-alkylbenzene-1,2-diol derivative or a 5-alkylbenzene-1,2,3-triol derivative.
15. The adhesive of any preceding embodiments wherein W has a structure selected from:

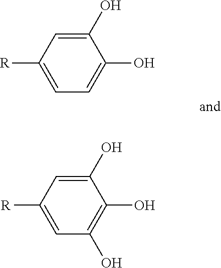

wherein R is present or absent and when present is a C1-C6 alkyl group or C1-C6 alkene optionally substituted with OH, NH.
16. The adhesive of any preceding embodiments comprising a compound having m is 1 and n is 0 and another compound having m is 1 and n is 0, 1, 2, 3 or 4.
17. The adhesive of any one of embodiments 1 to 15 comprising a compound having m is 1 and n is 0 and another compound having m is 1 and n is 1.
18. The adhesive of any preceding embodiments consisting or consisting essentially of one or more compounds of structure 1 or a cross-linked polymer produced by cross-linking compounds of structure 1 or wherein one or more compounds of structure 1 or a cross-linked polymer produced by cross-linking compounds of structure 1 are the sole adhesive compounds in the adhesive.
19. The adhesive of any preceding embodiments comprising a cross-linked gel.
20. The adhesive of any of embodiments 1 to 18 having a viscosity sufficient to be expelled out of a syringe at a temperature of at least 20° C.
21. The adhesive of any of embodiments 1 to 18 having an adhesive strength greater than 25 KPa under compression, tensile, or tensile lap shear testing.
22. The adhesive of any of embodiments 1 to 18 wherein the adhesive has free-flowing behavior at a temperature of at least 20° C.
23. The adhesive of any preceding embodiments further comprising a biological agent, pharmaceutical agent or diagnostic agent covalently bound or complexed to the compound or entrapped within the adhesive.
24. The adhesive of any of embodiments 1 to 17 further comprising a biocompatible polymer or oligomer, preferably a biodegradable polyester.
25. The adhesive of embodiment 24, wherein the adhesive comprises 5-95%, preferably >50 to 95%, by weight compounds of structure 1 and 5-95% by weight of a biocompatible polymer or oligomer.
26. The adhesive of any preceding embodiments, wherein the compound is an amorphous solid.
27. A method of adhering a first surface and a second surface comprising: applying a water soluble adhesive of preceding embodiments neat to at least a portion of the first surface; and bringing at least a portion of the second surface into contact with the adhesive.
28. The method of embodiment 27 wherein at least one of the surfaces is a surface contaminated with proteins and/or biomolecules.
29. The method of embodiment 27 or 28 wherein the first surface comprises a tissue and the second surface comprises a surface of an implant or device.
30. The method of any of embodiments 27 to 29 further comprising applying an external energy source to the adhesive and/or at least one of the first and second surface to enhance adhesion or application.
31. The adhesive of embodiment 1 wherein the compound is prepared by reacting a first isocyanate group of a diisocyanate having a substituted or unsubstituted alkyl-derived chain extender, a substituted or unsubstituted cycloalkyl-derived chain extender, or a substituted or unsubstituted benzyl-derived chain extender between the two isocyanate groups
with terminal amino or alcohol groups of an oligomer having at least two terminal amino or alcohol groups and
reacting a second isocyanate of the diisocyanate molecule with an amine group, alcohol group or carboxy group present on an alkyl chain of a 4-alkylbenzene-1,2-diol derivative or a 5-alkylbenzene-1,2,3-triol derivative.
32. The adhesive of embodiment 31 wherein the oligomer is an oligomer derived from a polyether.
33. The adhesive of embodiment 31 or 32 wherein the diisocyanate molecule is selected from the group consisting of:

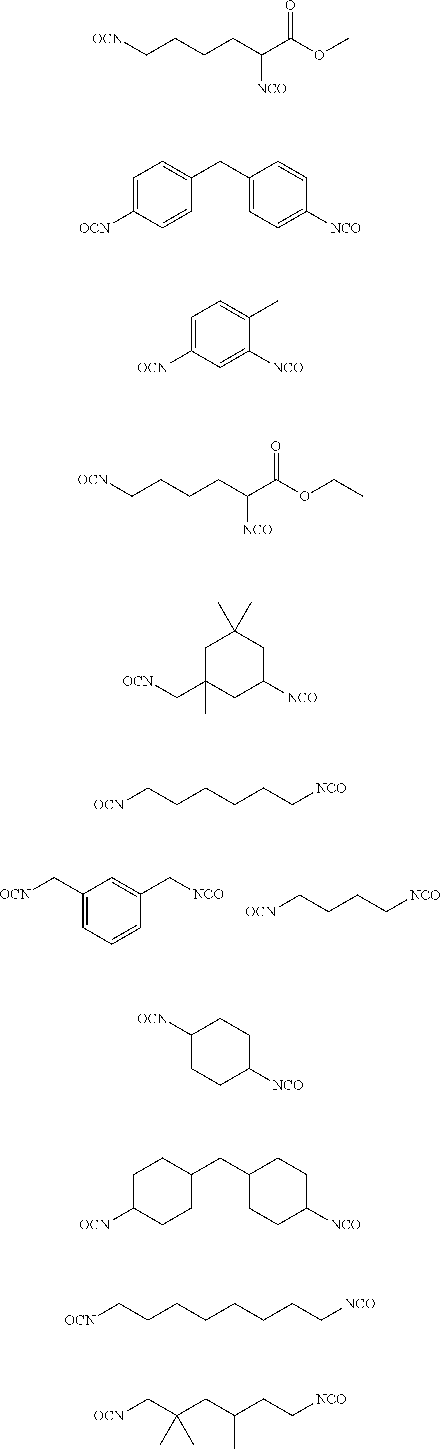

34. The adhesive of any of embodiments 31 to 33 wherein the 4-alkylbenzene-1,2-diol derivative or a 5-alkylbenzene-1,2,3-triol derivative is selected from the group consisting of:

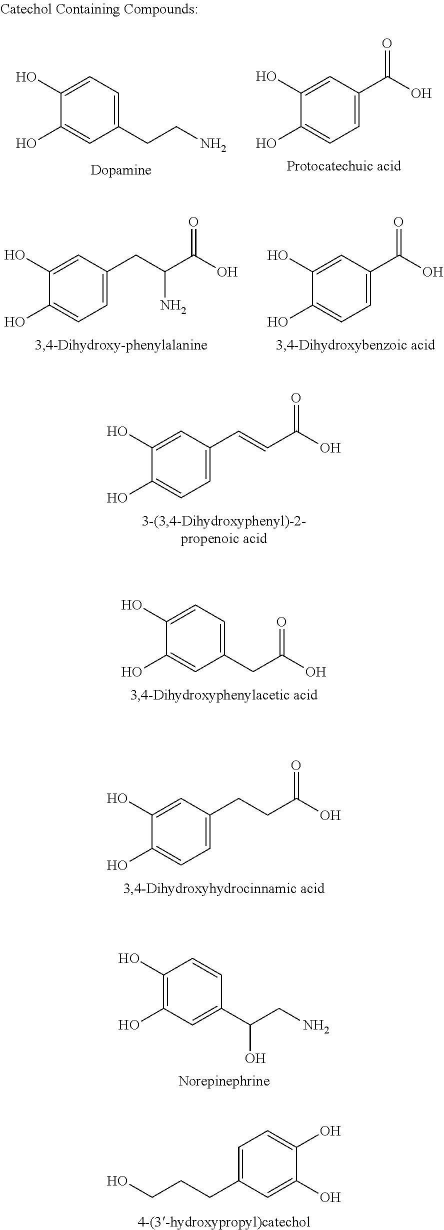

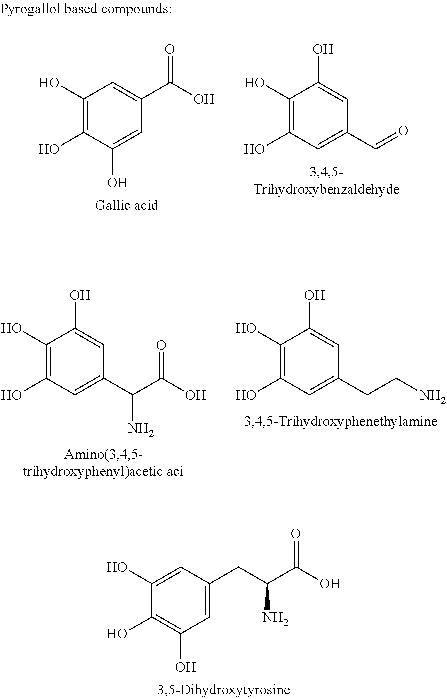 [SMILES: O=C(O)c1cc(O)c(O)c(O)c1 ; O=Cc1cc(O)c(O)c(O)c1 ; NC(C(=O)O)c1cc(O)c(O)c(O)c1 ; NCCc1cc(O)c(O)c(O)c1 ; N[C@@H](Cc1cc(O)c(O)c(O)c1)C(=O)O]

In other embodiments, there is provided any novel water soluble compound of structure 1 as provided above or as exemplified in the present description.

### BRIEF DESCRIPTION OF THE DRAWINGS

These and other features of the preferred embodiments of the invention will become more apparent in the following detailed description in which reference is made to the appended drawings wherein:

FIG. 1 shows compound of Structure 1 according to an embodiment;

FIG. 2 shows compound of Structure 2 according to an embodiment;

FIG. 3 shows compound of Structure 3 according to an embodiment;

FIG. 4 shows compound of Structure 4 according to an embodiment;

FIG. 5 shows compound of Structure 5 according to an embodiment;

FIG. 6 shows compound of Structure 6 according to an embodiment;

FIG. 7 shows compound of Structure 7 according to an embodiment;

FIG. 8 shows compound of Structure 8 according to an embodiment;

FIG. 9 shows an exemplary list of polyalcohols and sugar alcohols that may be used as a starting point for the preparation of the oligomer described herein;

FIG. 10 shows the general preparation of PPG pentaol oligomers according to any embodiment;

FIG. 11 shows an exemplary list of diisocyanate molecules that may be used for the preparation of compounds described herein;

FIG. 12 shows an exemplary list of 4-alkylbenzene-1,2-diol derivative and a 5-alkylbenzene-1,2,3-triol derivative that may be used for the preparation of compounds described herein;

FIG. 13 shows a method for the activation of dopamine hydrochloride into free dopamine;

FIG. 14 shows a generalized reaction scheme for the synthesis of trifunctional PCL adhesives used in the examples;

FIG. 15 shows a generalized reaction scheme for the synthesis of hexafunctional PCL adhesives via tetrakis(hydroxymethyl)oxydipropanol used in the examples;

FIG. 16 shows a generalized reaction scheme for the synthesis of difunctional PPG adhesive according to an embodiment;

FIG. 17 shows a generalized reaction scheme for the synthesis of difunctional PEG adhesive according to an embodiment;

FIG. 18 shows a synthesis schematic for water soluble adhesive 3 that uses a polyethylene glycol with three functionality of alcohol groups; according to an embodiment; and

FIG. 19 shows the affect of residual solvent on adhesive strength.

### DETAILED DESCRIPTION

For further clarity, the following terms are defined:

Tm (Melting temperature) is defined as the temperature (or temperature range) at which a well defined first order transition occurs, as determined by an endothermic event in a differential scanning calorimetry thermogram. If there is no such transition, as is common for amorphous materials, Tm will be defined by the beginning of the substance's ability to flow or spread under an applied force (i.e., temperature within the linear viscoelastic region on a stress-sweep viscoelastic curve at which the viscous modulus>elastic modulus (tan δ>1)).

Tg (Glass transition temperature) is defined as the temperature (temperature range) where the polymer transitions from a hard, glassy material to a soft, rubbery material as determined by changes in the heat capacity (Cp), reversible heat flow curves, and material rigidity or hardness obtained from differential scanning calorimetry, dynamic mechanical assessment and needle penetrometer or indentation experiments, respectively.

Physiological conditions refer to wet conditions at pH 7.4±0.3 and temperature of 37.5±3° C.

Water soluble is defined as the absence of visible particulates and/or the formation of a homogeneous mixture when a solvent-free material is hand mixed with water at a concentration of 1.0 wt/wt % at physiological temperatures (37.5±3° C.).

In some embodiments, adhesive compounds as provided herein are water soluble i.e. there is an absence of visible particulates and/or formation of a homogeneous mixture when a solvent-free material is mixed with water up to a concentration of 40 wt/wt %.

Crosslinking is defined as the formation of a chemical bond, inclusive of covalent bonding and chelation, between two existing macromolecules (for example: adhesive-adhesive crosslinking, adhesive-protein crosslinking).

Curing is defined as the chemical process of converting a macromolecule into a higher molecular weight polymer via crosslinking reactions.

In the present disclosure, soft tissue refers to connective and/or fatty and/or fibrous soft tissues and/or any organ tissue consisting primarily of low calcified content relative to biomolecule content (e.g. collagens, glycosamino glycans, elastin, etc). Soft tissues connect, surround, or support bones, organs and other structures in the body. Examples of soft tissues include tendons, muscles, skin, ligaments, nerves, vessels, fascia, fibrous tissues and synovial membranes.

In the present disclosure, hard tissues are mineralized and rigid tissues containing significant amounts of hydroxyapatite. Examples of hard tissues include bone, enamel, dentin and cementum.

Implant is defined as any material that is designed to function in contact with the human body and can include topical implants such as for application to body surfaces such as the skin or mucous membranes.

The present disclosure provides an adhesive that may be used to glue or secure tissue to implant or implant to implant.

Many previously described adhesives are comprised of polymeric chains that are decorated with periodic or randomly distributed branching side chains containing adhesive functional groups. This contributes to high intermolecular bonding (requiring solvents to assist with application and surface wetting) and poor accessibility of adhesive functional groups to the substrates where adhesion is desired.

Structures described in the present disclosure when compared to known bioadhesives may be characterized by a rapid high degree of adhesion to a biological substrate, easy adherence and retention of the adhesive to substrates, and/or elimination of solvent requirement.

The Examples evidence that catechol-based biomedical adhesives can be prepared based on low molecular oligomers that do not require solvent for application. Further, cross-linking can be used to increase the adhesive strength of these adhesive compounds thereby yielding useful compositions without the need for high levels of filler. Further, the examples evidence that the presence of residual solvent interferes with cross-linking.

### Water Soluble Adhesive Compounds

The present disclosure describes adhesive compounds consisting of short linear or short branched oligomer chains capped only at their terminal ends with phenyl moieties with two or three hydroxyl groups acting as adhesive moieties. The oligomer chains may have a MW between 100 g/mol or 500 g/mol and 3000 g/mol; in one embodiment between 100 g/mol, 500 g/mol and 2000 g/mol; in one embodiment, between 100 g/mol, 500 g/mol and 1000 g/mol.

The present disclosure provides adhesives which are biocompatible and non-toxic; wherein biocompatible refers to a material which does not induce an observable biological response (e.g. immunological) upon contact with cellular, tissue or other bodily fluids/components [20], and non-toxic refers to a material which does not negatively affect cell viability. Furthermore, when the compounds of the adhesive are tailored to be degradable, the degradation products are biocompatible and non-toxic.

While not wishing to be bound by theory, having moieties at the terminal ends allows the adhesive moieties to preferentially orient to the substrate surface with minimal steric hindrance, and therefore rapid adhesion. Terminal functionalization of adhesive moieties onto the oligomeric backbone may also allow structural control, as many internally functionalized block or grafted polymers have adhesive moieties randomly oriented off of the backbone. Terminal grafting may also reduce the viscosity of the adhesives, as their bulk disrupts packing, reducing intermolecular forces.

In addition, having oligomer chains with a Mw<5,000 g/mol, preferably <3,000 g/mol and in some embodiments <1000 g/mol greatly reduces intermolecular interactions associated with them due to smaller size and reduced cumulative forces between oligomers. The reduction in intermolecular interactions between the oligomer chains of the compound of structure 1 can allow the adhesive of the present disclosure to be prepared without the use of a solvent resulting in faster setting/curing, low/no toxicity and improved usability of the adhesive, as solvent has been reported to interfere with adhesive bonding and presents its own toxicity concerns [21-24].

It is well accepted that adhesives formulated for use in biomedical applications ideally may have some or many of the following characteristics [25-30]: bond rapidly or on demand when in contact with a surface; cure in a controllable manner; work over a suitable range of temperatures; adhere to materials without the necessity of pre-treatment or cleaning; exhibit adhesion strength which is maintained over the duration of the desired application; have strong bonding and curing strength; are biodegradable; obviate the need for a solvent; achieve desirable aesthetic properties; are easy to work with and easy to apply; easy to incorporate a catalyst to achieve curing, or cures without catalyst; are easy and inexpensive to manufacture; work well in situations where contaminants may be present. Depending on the structure, the Mw, and the formulation of the compounds used in the present disclosure, the adhesives described in the present disclosure may have at least one of the criteria listed above.

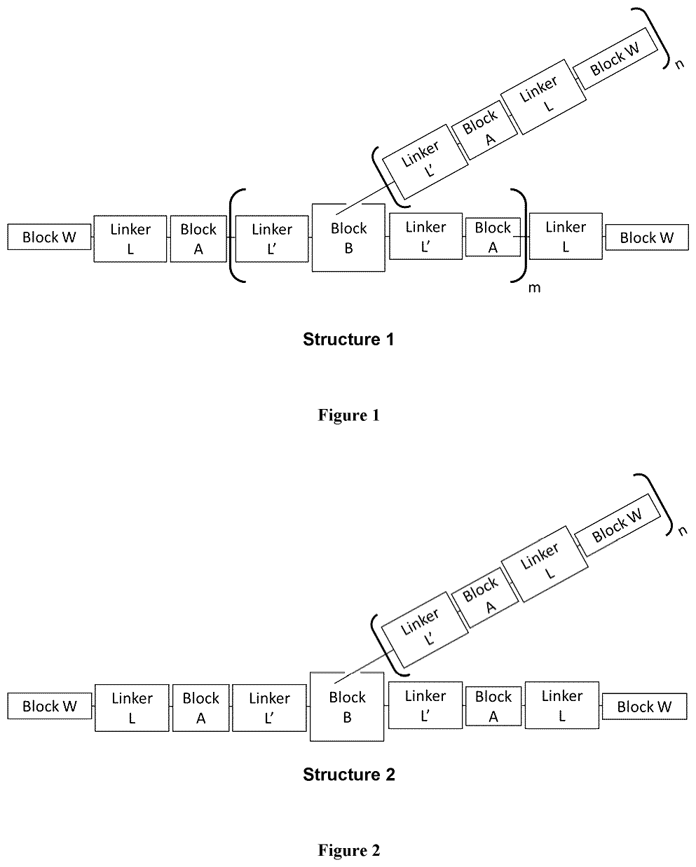

According to an embodiment, an adhesive is described. The adhesive comprises, consists of or consists essentially of a compound of structure 1 (FIG. 1) wherein B is a branched or unbranched oligomer, Linker L is a urethane, urea bond, or amide bond; Linker L′ is a urethane or urea bond, A is a chain extender, W is a terminal adhesive moiety, m is 0 or 1; and n is 0, 1, 2, 3 or 4.

Water solubility can be rendered by assembling an adhesive using B and A components, and W components, which each themselves may or may not be water soluble.

According to an embodiment, the adhesive comprises one or more water soluble compounds of the structure 1. The adhesive can be rendered less water soluble by adding a suitable crosslinking agent such that the soluble adhesive undergoes a chemical reaction in the presence of the crosslinking agent. Thus, the adhesive may be prepared in such way that it will dissolve over a limited and/or specific period of time when exposed to physiological conditions. In one embodiment, the adhesive itself is water soluble as defined above.

According to an embodiment, the compound of structure 1 has m=1 resulting in the compound of structure 2 (FIG. 2).

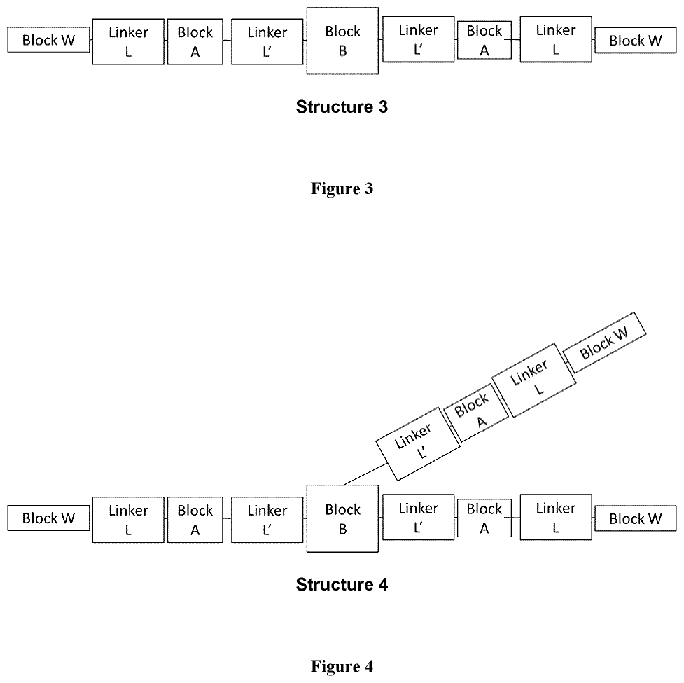

According to an embodiment, the compound of structure 1 has m=1 and n=0 resulting in the compound of structure 3 (FIG. 3).

According to an embodiment, the compound of structure 1 has m=1 and n=1 resulting in the compound of structure 4 (FIG. 4).

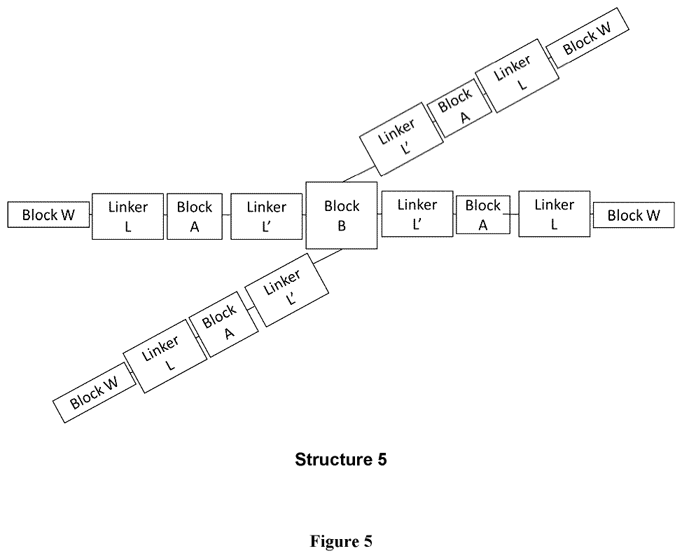

According to an embodiment, the compound of structure 1 has m=1 and n=2 resulting in the compound of structure 5 (FIG. 5).

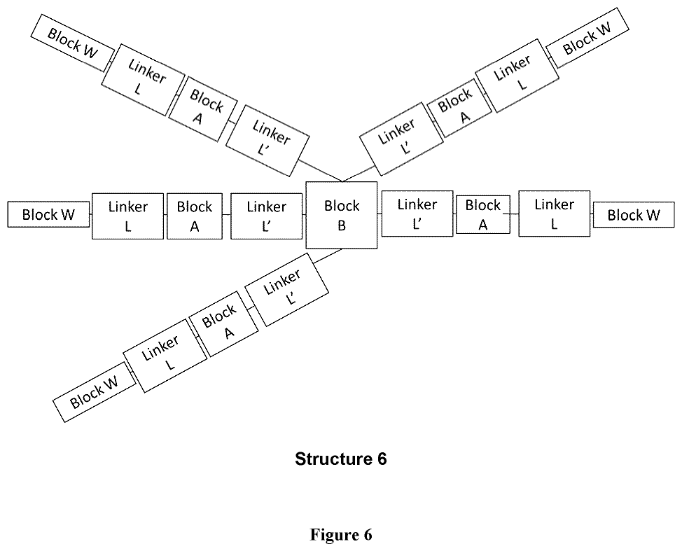

According to an embodiment, the compound of structure 1 has m=1 and n=3 resulting in the compound of structure 6 (FIG. 6).

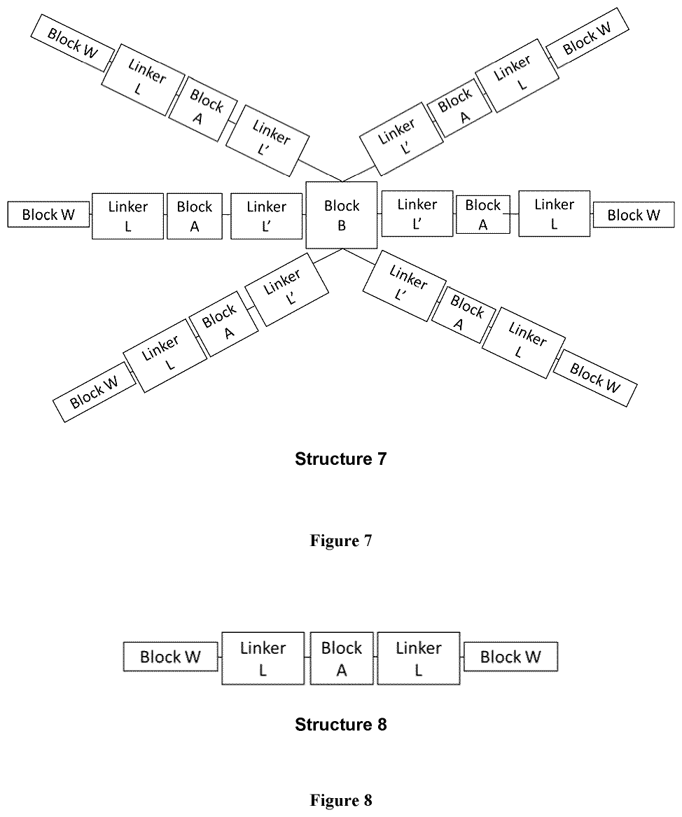

According to an embodiment, the compound of structure 1 has m=1 and n=4 resulting in the compound of structure 7 (FIG. 7).

Alternatively, according to an embodiment, the compound of structure 1 has m=0 resulting in the compound of structure 8 (FIG. 8).

Block B is an oligomer having a MW between 50 g/mol and 5,000 g/mol, In various embodiments, between 100 g/mol or 500 g/mol and 1000 g/mol, 2000 g/mol, 3000 g/mol. Preferably, the oligomer chain may have a Mw<3,000 g/mol. The Block B may be derived from any suitable biocompatible polymer, which can include, polysaccharides, polyamides, polyethers, or polyalkylene glycols In one embodiment, Block B is derived from a polyether or polyalkylene glycol. According to some embodiments, the Block B is an oligomer derived from polypropylene glycol or polyethylene glycol. As used herein, an oligomer “derived” from an identified polymer retains the essential structure and activity of the polymer despite any modifications thereto. In various embodiments, the oligomer chain is selected from the identified biocompatible polymers.

Block B may have a finite number of branches resulting in compounds of Structure 1 or Structure 2 or more particularly compounds of Structure 4, Structure 5, Structure 6 or Structure 7. Alternatively, Block B may be linear resulting in compounds of Structure 3.

According to an embodiment, Block B may be derived from small molecules such as monomers, dimers or trimers. Such monomers, dimers and trimers may be derived from polyhydric alcohols including but not limited to trimethylolpropane, glycerol and pentaerythritol; or from their alkoxylated derivatives.

According to an embodiment, the adhesive of the present disclosure may be a compound of structure 8 for which Block B is absent.

Linker L is a urethane bond, a urea bond or an amide bond and Linker L′ is either a urethane bond or a urea bond. According to an embodiment, the compound of structure 1 may be designed to render Linker L and/or Linker L′ hydrolysable or enzyme degradable.

Block A is a chain extender comprising substituted or unsubstituted alkyl, cycloalkyl and/or aromatic groups. Block A has a Mw between 20 and 3000 g/mol in some embodiments, between 20 and 65 g/mol, 100 g/mol, 200 g/mol, 500 g/mol, 1000 g/mol or 2000 g/mol. According to an embodiment, Block A is a substituted or unsubstituted alkyl group. According to an embodiment, Block A is a substituted alkyl group, wherein one or more of the hydrogens on the alkyl are replaced, introducing branching. According to an embodiment, Block A may be substituted with C1-C6 carboxylate group. According to another embodiment, Block A is a methyl hexanoate or ethyl hexanoate.

Block W is a terminal adhesive moiety having a phenyl with at least two hydroxyl groups. According to an embodiment, Block W is a terminal adhesive moiety derived from benzene-1,2-diol derivative or benzene-1,2,3-triol derivative. Biologically derived and inspired molecules containing benzene-1,2-diol or benzene-1,2,3-triol derivatives have strong and durable adhesion to various substrate surfaces including wet surfaces. These characteristics are advantageous for the preparation of adhesive molecules for biomedical applications. According to an embodiment, Block W may be a 4-alkylbenzene-1,2-diol derivative. Alternatively Block W may be a 5-alkylbenzene-1,2,3-triol derivative. According to an embodiment, Block W may have a structure selected from either Structure 9 or Structure 10 wherein R may be present or absent. When R is present, R may be a C1-C6 alkyl group or C1-C6 alkene optionally substituted with OH, NH or C1-C6 alkyl.

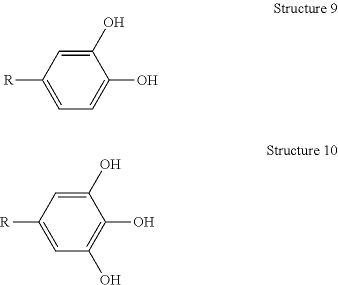

The adhesives described in the present disclosure comprise a compound suitably have a Tg lower than or equal to 25° C., in one embodiment lower than or equal to 20° C. According to an embodiment, the adhesives may have a viscosity sufficient to be expelled out of a syringe at a temperature of at least 20° C. Alternatively, the adhesive may have a viscosity sufficient to be expelled from a needle having a gauge between 7 gauge to 33 gauge or more specifically 16 gauge, 17 gauge or 21 gauge at a temperature of at least 20° C. and less than 50° C. According to another embodiment, the adhesive of the present disclosure may have a free-flowing behavior at a temperature of at least 20° C.

According to an embodiment, the adhesives described in the present disclosure may have an adhesive strength greater than 10 kPa under compression, tensile, or tensile lap shear testing. According to other embodiments, the adhesives described in the present disclosure may have an adhesive strength greater than 25 kPa, 50 kPa, greater than 75 kPa or greater than 100 kPa under compression, tensile, or tensile lap shear testing. Adhesives according to disclosed embodiments that have an adhesive strength between 10 kPa and 25 kPa may have particular utility in applications where it is beneficial to have an adhesive that is relatively flexible, including applications involving the adhesion of soft tissues and, in particular, skin.

Compared to the starting oligomers, the adhesives possess increased hydrophilicity on account of the urethane and urea linkages present in the adhesives, in addition to the already existing ether group linkages of the starting oligomers. These properties make the adhesives susceptible to water ingress during use.

According to an embodiment, a method of preparing the compound of structure 1 is provided. The method comprises the steps of:

1. reacting an amino or alcohol group present at each of at least two end portions of an oligomer chain with one of two isocyanate groups of a diisocyanate molecule. The diisocyanate has a substituted or unsubstituted alkyl-derived chain extender, a substituted or unsubstituted cycloalkyl-derived chain extender, or a substituted or unsubstituted benzyl-derived chain extender sandwiched between the two isocyanate groups;
2. reacting the second isocyanate of the diisocyanate molecule with an amine group, alcohol group or carboxy group present on an alkyl chain of a 4-alkylbenzene-1,2-diol derivative or a 5-alkylbenzene-1,2,3-triol derivative.

According to another embodiment, a method of preparing the compound of structure 8 is provided. The method comprises the step of reacting each isocyanate group of a diisocyanate with an amine group, alcohol group or carboxy group present on an alkyl chain of a 4-alkylbenzene-1,2-diol derivative or a 5-alkylbenzene-1,2,3-triol derivative. The diisocyanate has a substituted or unsubstituted alkyl-derived chain extender, a substituted or unsubstituted cycloalkyl-derived chain extender, or a substituted or unsubstituted benzyl-derived chain extender between the two isocyanate groups.

According to an embodiment, the oligomer backbone has a Mw of about <5,000 g/mol or <3,000 g/mol or ˜ 1000 g/mol. The oligomer chain may be non-degradable. Alternatively, the oligomer chain may be hydrolysable or enzyme degradable under physiological conditions. In one embodiment, the oligomer may be derived from any suitable polymer currently used in implant devices and known to those skilled in the art. In one embodiment, the oligomer is derived from a polyether, or polyalkylene glycol. In preferred embodiments, the oligomer may be derived from polypropylene glycol or polyethylene glycol.

According to an embodiment, the oligomer may be linear, resulting in compounds of Structure 3. Alternatively, the oligomer may be branched resulting in compounds of Structure 4, Structure 5, Structure 6 or Structure 7.

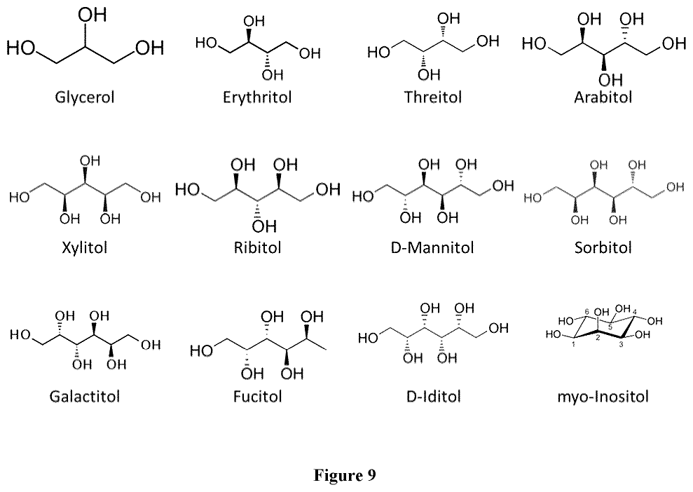

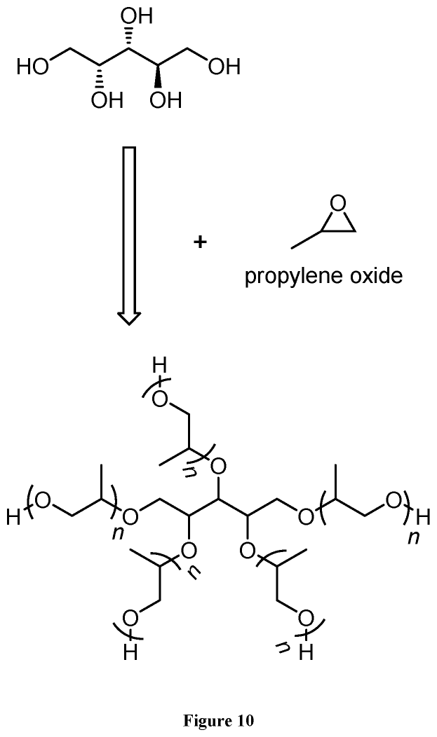

According to an embodiment, the oligomer may be selected from commercially available oligomers. Alternatively, the oligomers may be prepared by a skilled person using common general knowledge in the field of polymer chemistry [31]. FIG. 9 shows non-limiting examples of sugar alcohols that may be used as a starting point for the preparation of the oligomer. FIG. 10 shows an example of the general preparation of PPG pentaol oligomers.

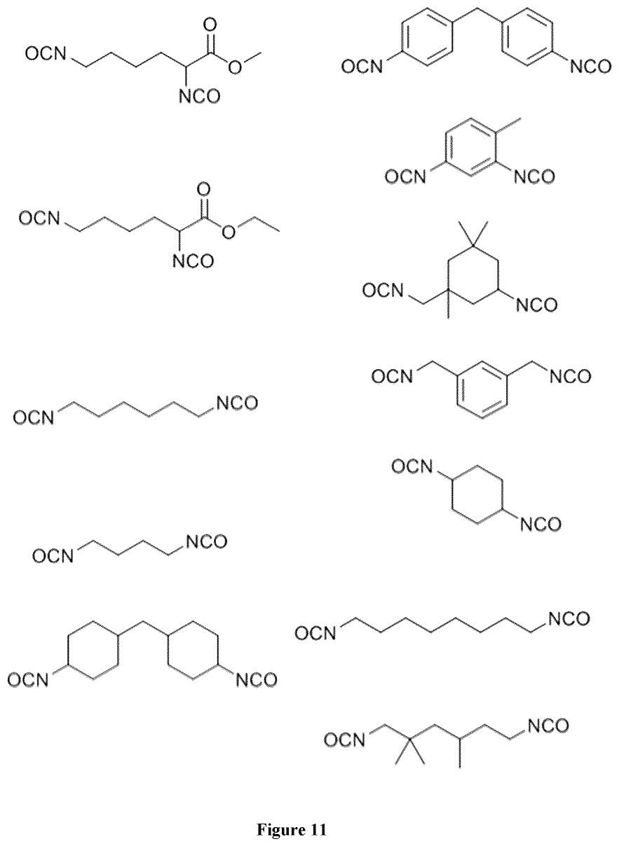

According to an embodiment, the diisocyanate molecule may be selected from the group shown in FIG. 11.

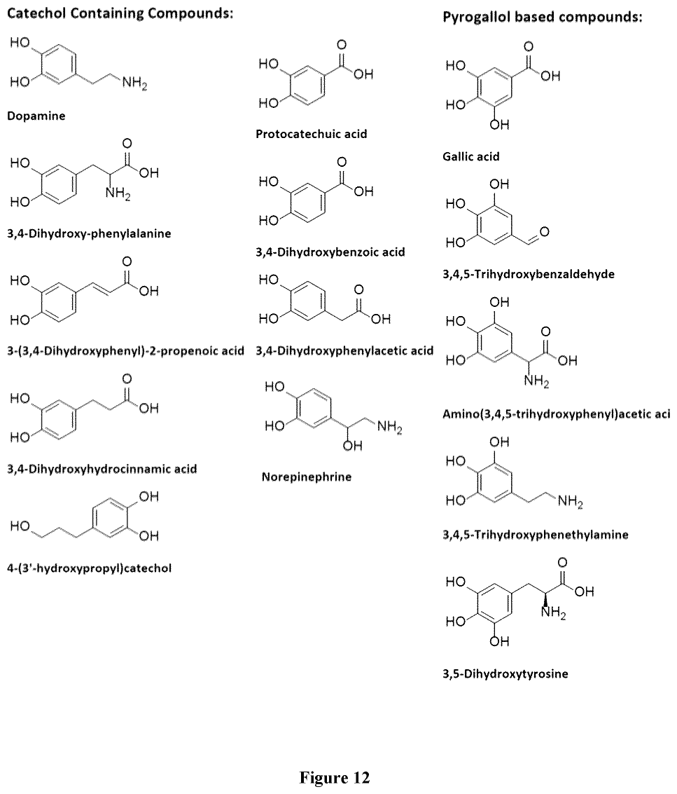

According to an embodiment, the 4-alkylbenzene-1,2-diol derivative or a 5-alkylbenzene-1,2,3-triol derivative may be selected from the group shown in FIG. 12. This class of compounds have been known for their adhesion properties and are suitable for the synthesis of adhesive molecules. According to another embodiment, the preferred 4-alkylbenzene-1,2-diol derivative is dopamine and the preferred 5-alkylbenzene-1,2,3-triol derivative is 3,4,5-trihydroxyphenethylamine.

### Adhesives

According to an embodiment, the adhesives of the present disclosure may comprise a plurality of compounds derived from the compound of structure 1. The adhesives may comprise a compound of structure 1 having m=0 and another compound of structure 1 having m=1 and n is either 0, 1, 2, 3 or 4. More particularly, the adhesives may comprise a compound of structure 1 having m=0 and another compound of structure 1 having m=1 and n is 0.

According to an embodiment, the adhesives of the present disclosure may comprise a compound of structure 1 having m=1 and n=0 and another compound of structure 1 having m=1 and n is 0, 1, 2, 3 or 4. More particularly, the adhesives may comprise a compound of structure 1 having m=1 and n=0 and another compound of structure 1 having m=1 and n=1. According to another embodiment, the adhesives of the present disclosure may consist or consist essentially of compounds of structure 1.

According to an embodiment, the adhesives of the present disclosure may comprise a plurality of compounds derived from the compound of structure 1 with additional admixture(s) whose function may include directly enhancing the adhesive performance (for example, but not limited to polymers, crosslinking agents, fillers and porogens), and/or to provide combination products with further value add (for example, but not limited to drugs, fillers, therapeutics, regenerative materials or other compounds).

In one embodiment, water soluble compound(s) of structure 1 or a polymer formed by cross-linked compound(s) of formula 1 comprise >50% by weight of the adhesive. In some embodiments, >60% by weight, >70% by weight, >80% by weight, >90% by weight or greater than 95% by weight of the adhesive. In some embodiments, the adhesive comprises between 90 and 99.5% by weight water soluble compound(s) of structure 1 with the remainder being cross-linker or curing agent. In some embodiments, the adhesive comprises between 0.1 and 10%, in one embodiment between 0.1 and 5% by weight curing or cross-linking agent.

Suitable crosslinking agents can include inorganic oxidants (Na₃VO₄ and tetrabutylammonium (meta)periodate (TBAP)), an organic or inorganic bases ((Na₂CO₃), triethylamine) or organic complexing salts (Iron (III) citrate—FeCit), FeCl₃ and FePO₄).

According to an embodiment, the adhesives of the present disclosure may further comprise a crosslinking agent or a curing agent, in order to achieve a desirable mechanical property and/or desired setting speed. In various embodiments, the setting speed may be ≤5 hours, ≤2 hours, ≤60 minutes, ≤15 minutes, ≤5 minutes, ≤2 minutes, ≤1 minute, ≤30 seconds. The use of such agents may improve the rigidity and cohesive strength of the adhesives while reducing creep and deformation. 4-alkylbenzene-1,2-diol derivative or a 5-alkylbenzene-1,2,3-triol derivative may be irreversibly crosslinked via the use of oxidants, light, basic pH, and enzymes. The examples demonstrate effective cross-linking agents for oligomer chains capped at their terminal ends with adhesive moieties described herein In some embodiments, the compounds of Structure 1 may have a lower Tg or melting point than the operational temperature. Under such conditions, a curing agent or crosslinking agent is useful to rigidify the adhesives, providing increased cohesive strength. When an oxidant is included within the adhesive, the oxidant may promote covalent crosslinking of the catechol groups with other catechols and/or tissue.

According to an embodiment, the adhesives described in the present disclosure comprise compounds of structure 1 having a Mw<5,000 g/mol, preferably <3000 g/mol and a Tg lower than 20° C. rendering optional the use of a solvent. According to an embodiment, the adhesives of the present disclosure may be solvent free. The solvent-optional or solvent-free nature of the adhesive of the present disclosure is advantageous as numerous solvents are known to be toxic and may impede with the bonding of the adhesive to the substrate and may even interfere with the crosslinking and curing process if crosslinking agents or curing agents are used. Adhesives dissolved in solvents have reduced cohesive and adhesive properties and rely on diffusion or evaporation of the solvent for improved mechanical performance. Depending on the environment and the nature of the solvent, this diffusion or evaporation of the solvent may be on the order of minutes, hours, days or even longer periods of time. Solvents have been known to cause toxicity or other undesirable effects including intoxication and strong odors [31-32]. Furthermore, using an adhesive that does not require a solvent is advantageous for biomedical applications as it alleviates the need for the solvent to evaporate, diffuse or be removed by another mechanism, which are frequent requirements when an adhesive system includes a solvent. Solvent removal may be accomplished in a number of ways, but most often is accomplished via evaporation or diffusion. These events are usually accomplished over an extended time period that may occur over hours or even multiple days, creating very long setting times and limiting the biomedical applications and use of these adhesive formulations.

### Applications of Adhesives

According to an embodiment, the adhesive of the present disclosure may further be combined with a biological agent, pharmaceutical agent or diagnostic agent covalently bound or complexed to the compound or entrapped within the adhesive. This could include a drug, protein, enzyme, combination, diagnostic reagent, regenerative material or biosensor. The agent may either diffuse out through the adhesive or be released as the adhesive dissolves.

According to an embodiment, the adhesives described in the present disclosure may be used by adhering a first surface and a second surface by applying the adhesive to at least one of the first and second surfaces; and bringing at least a portion of the first and second surfaces into contact with each other. According to another embodiment, the adhesive of the present disclosure may be used by applying it neatly to one or both of the first and second surfaces. The first surface may comprise soft tissue and the second surface may comprise hard tissue such as bone. Alternatively, both surfaces may be soft tissue, or hard tissue such as bone. Alternatively, the first surface may comprise soft tissue and the second surface may be a surface of an implant or device. Alternatively, the first surface may comprise hard tissue such as bone and the second surface may be a surface of an implant or device.

According to an embodiment, the adhesive can be applied to the above surfaces using a source of external energy to enhance the curing of the adhesive with said surface.

In one embodiment, the adhesive itself may function as an implant such as where the adhesive may be rendered sufficiently rigid so as to permit it to be shaped for a particular medical purpose e.g. it may be used to encapsulate an agent that will be released when the adhesive dissolves.

Non-limiting examples of implantable devices that could benefit from attachment in situ using the bioadhesives include lead for real-time glucose monitoring meter, lead for a pacemaker, and insulin delivery system.

According to an embodiment, the present disclosure also provides a device comprising an adhesive having at least one compound of structure 1. The device comprises the adhesive and a support, in one embodiment, a biocompatible support. The support may, for example, be a sheet structure and the adhesive may be coated onto or impregnated into the sheet or portions thereof (e.g. there may be discrete bonding sites). In one embodiment, the sheet is a flexible rigidifiable biocompatible sheet. In one embodiment, the adhesive is located on a first surface of the sheet. In one embodiment, the sheet has first and second opposed surfaces and both surfaces are coated with the adhesive. In one embodiment, the main constituent on the biocompatible sheet structure is a biodegradable and/or bioresorbable polymer.

Adhesive may be applied to supports by methods known to those of skill in the art and can include e.g. spraying the adhesive onto the support or submerging the adhesive in the support (for applications where all surfaces of the support will be coated with adhesive). The adhesive may be cross-linked to the support. In some embodiments, cross-linking reactions are delayed until time of use e.g. by storing the device under conditions that inhibit cross-linking reactions. Various approaches are available to ensure stability of the devices and can include cold storage and transportation, addition of antioxidants, encapsulation of the cross-linking agents in temperature triggered materials, careful selection of cross-linking agents that do not react at common storage conditions or addition of cross-linking agents that require external energy to initiate the cross-linking reactions.

The support material must have compatibility with the adhesive such that adhesion between the adhesive and support are sufficient to provide application specific performance over a relevant timeline. Incompatible support materials may be chemically or physically modified to improve adhesion by treatment with oxygen plasma, acid etchants, basic agents, binding agents or other suitable surface modifiers.

The device may be an implant comprising a biocompatible support and an adhesive as described herein coated onto or impregnated into the support.

The support may be hydrolysable or enzyme degradable under physiological conditions.

In one embodiment, the adhesive may be incorporated into or coated onto a patch or tape for use in medical procedures.

In some embodiments, the adhesives described herein are suitably used as external bioadhesives, including, in particular, for topical applications. According to an embodiment, the adhesive may be used for the fixation of medical devices to a target site. Examples may include fixation of a topical therapeutic patch a wound dressing or bandage, a sensor, a catheter or a graft.

According to an embodiment, the adhesive may be used to render a biomedical mesh adherent to tissues. In another embodiment, the adherent mesh may be loaded with a drug, protein, biomaterial or other therapeutic agent to enhance or improve healing.

According to an embodiment, the adhesive may be used to functionalize the surface of synthetic or natural biomaterials or devices. This may be used to improve cellular adhesion to the surface, control, select or recruit adhesion of preferential types of cells, or attach a therapeutic agent or compound to the surface to a synthetic or natural biomaterial or device. In a select embodiment, this could be used to adhere antibiotic or antifungal elements to a material, rendering the material less susceptible to bacterial or fungal contaminations. In another embodiment, the described bioadhesive may be used to functionalize a non-adherent surface so that cells or proteins are able to attach to the surface, enabling or improving surface functionalization and degree of integration.

According to an embodiment, the adhesive may be dissolved in a solution where it is subsequently cross-linked in solution using a cross-linking agent to form a hydrogel or other type of gel. This gel may encapsulate antibiotics, therapeutic or regenerative materials from the solution or may alternatively be impregnated with antibiotics, therapeutic or regenerative materials following formation. According to an embodiment, a cross-linking agent may be used to crosslink the adhesive in solution to form micro or nano sized particles, encapsulate antibiotics, therapeutic or regenerative materials from the solution or may alternatively be impregnated with antibiotics, therapeutic or regenerative materials following formation. Biocompatible cross-linkers are known to those of skill in the art and suitable cross-linking agents are exemplified herein.

In the applications described above the dissolution of the adhesive can be exploited e.g. for facilitating the delivery of active agents or as temporary adhesives that can be readily removed by washing.

All documents referenced herein are incorporated by reference, however, it should be appreciated that any patent, publication, or other disclosure material, in whole or in part, that is incorporated by reference herein is incorporated only to the extent that the incorporated material does not conflict with definitions, statements, or other disclosure material set forth in this disclosure. As such, and to the extent necessary, the disclosure as explicitly set forth herein supersedes any conflicting material incorporated herein by reference.

The above description and accompanying drawings should be taken as illustrative of the invention and it is to be understood that it is intended to cover any variations, uses, or adaptations of the invention following, in general, the principles of the invention and including such departures from the present disclosure as come within known or customary practice within the art to which the invention pertains. The embodiments of the invention described above are intended to be exemplary only. The scope of the invention is therefore intended to be limited solely by the scope of the appended claims.

### Example 1: Trifunctional PCL Adhesive (Adhesives 1a-c) (Non-Water Soluble Adhesive Compounds)

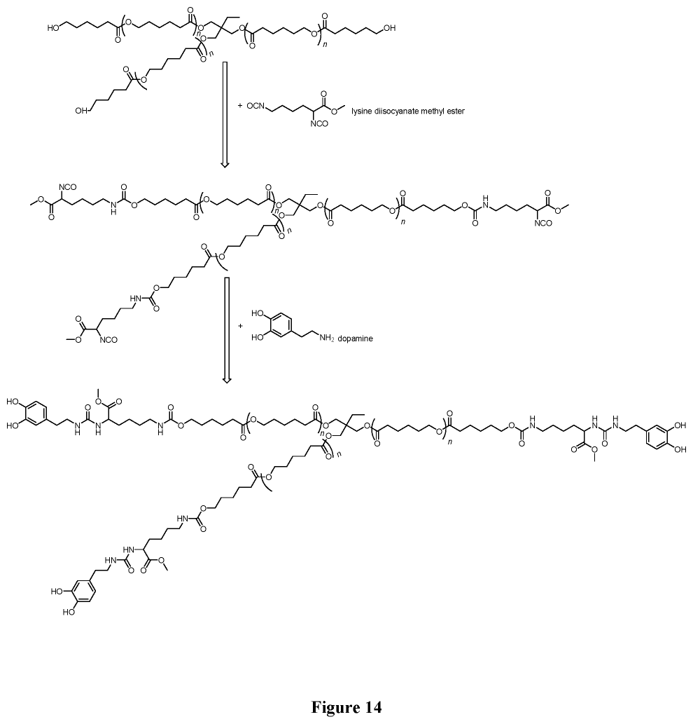

FIG. 14 shows a generalized reaction scheme for the synthesis of trifunctional PCL adhesives in which the terminal hydroxyl groups of a PCL-triol reacts with one of two isocyanate groups of the diisocyanate molecule resulting in a PCL-triisocyanate molecule. The free isocyanates then react with a reactive moiety (here an amine) present on the alkyl chain of the 4-alkylbenzene-1,2-diol derivative (here dopamine)

### Example 1a: Synthesis of Adhesive 1a as an Example of a Trifunctional PCL Adhesive

### a) Activation of the Dopamine Derivative (Dopa):

Dopamine hydrochloride was activated as previously described. One molar equivalent of dopamine hydrochloride was dissolved into a minimum of anhydrous DMAc at room temperature. An equivalent amount of triethylamine was added to complex the hydrochloride salt from dopamine. The mixture was stirred for 30 minutes, yielding a cloudy solution. Triethylamine hydrochloride was separated from the activated dopamine by centrifuging for 10 minutes at 5000 rpm and −10° C. The activated dopamine was decanted from the solid triethylamine pellet and used immediately.

### b) Synthesis of Isocyanate Terminated PCL Oligomers (Prepolymer 1a):

Ten mmol of polycaprolactone triol with an average molecular weight of 300 g/mol (n=0.7) was added to a dry 250 mL 3-neck flask and degassed under vacuum at 60° C. for 12 h. The flask was purged with argon and 25 mL anhydrous DMAc was added through a septum with stirring under argon protection. Once the reaction mixture was completely clear, it was cooled to room temperature. 31.5 mmol of LDI (Methyl Ester L-Lysine Diisocyanate, 5% molar excess to hydroxyl groups) was added to the reaction mixture with a syringe through a silicone rubber septum and the mixture was stirred for 1 hour at room temperature. The reaction mixture was then ramped to 60° C. over 4 hours, followed by a second ramp to 75° C. over an additional 4 hours. Finally, the reaction was stirred at 75° C. for 2 hours, then cooled to room temperature to give prepolymer 1a.

### c) Synthesis of PCL Based Oligomeric Dopamine Adhesive (Adhesive 1a)

Prepolymer 1a was functionalized by adding 33 mmol of activated dopamine in 50 mL of DMAc to the reaction mixture. An ice bath was used to dissipate heat from this rapid and exothermic step. The reaction was stirred for an additional 2 hours to ensure complete reaction, then the mixture was slowly poured into stirred deionized ice water (200 mL) at 0° C. Stirring was ceased, and Adhesive 1a was recovered as a thick viscous fluid on the bottom of the flask after decanting the water fraction. It was re-dissolved in acetone (75 mL) and precipitated out of solution with deionized water (200 mL) twice more to remove any residual triethylamine, dopamine, LDI derivatives and other reaction by-products.

### Example 1b: Synthesis of Adhesive 1b as an Example of Trifunctional PCL Adhesive

### a) Activation of the Dopamine Derivative (Dopa):

Dopamine hydrochloride was activated as previously described. One molar equivalent of dopamine hydrochloride was dissolved into a minimum of anhydrous DMAc at room temperature. An equivalent amount of triethylamine was added to complex the hydrochloride salt from dopamine. The mixture was stirred for 30 minutes, yielding a cloudy solution. Triethylamine hydrochloride was separated from the activated dopamine by centrifuging for 10 minutes at 5000 rpm and −10° C. The activated dopamine was decanted from the solid triethylamine pellet and used immediately.

### b) Synthesis of Isocyanate Terminated PCL Oligomers (Prepolymer 1 b):

10 mmol of polycaprolactone triol with an average molecular weight of 900 g/mol (n̅=2.3) was added to a dry 250 mL 3-neck flask and degassed under vacuum at 60° C. for 12 h. The flask was purged with argon and 25 mL anhydrous DMAc was added through a septum with stirring under argon protection. Once the reaction mixture was completely clear, it was cooled to room temperature. 31.5 mmol of LDI (Methyl Ester L-Lysine Diisocyanate, 5% molar excess to hydroxyl groups) was added to the reaction mixture with a syringe through a silicone rubber septum and the mixture was stirred for 1 hour at room temperature. The reaction mixture was then ramped to 60° C. over 4 hours, followed by a second ramp to 75° C. over an additional 4 hours. Finally, the reaction was stirred at 75° C. for 2 hours, then cooled to room temperature to give prepolymer 1 b.

### c) Synthesis of PCL Based Oligomeric Dopamine Adhesive (Adhesive 1 b)

Prepolymer 1b was functionalized by adding 33 mmol of activated dopamine in 50 mL of DMAc to the reaction mixture. An ice bath was used to dissipate heat from this rapid and exothermic step. The reaction was stirred for an additional 2 hours to ensure complete reaction, then the mixture was slowly poured into stirred deionized water (200 mL) at room temperature. Stirring was ceased, and Adhesive 1b was recovered as a thick viscous fluid on the bottom of the flask after decanting the water fraction. It was re-dissolved in acetone (75 mL) and precipitated out of solution with deionized water (200 mL) twice more to remove any residual triethylamine, dopamine, LDI derivatives and other reaction by-products.

### Example 2: Hexafunctional PCL Adhesive (Adhesive 2) (Non-Water Soluble Adhesive Compound)

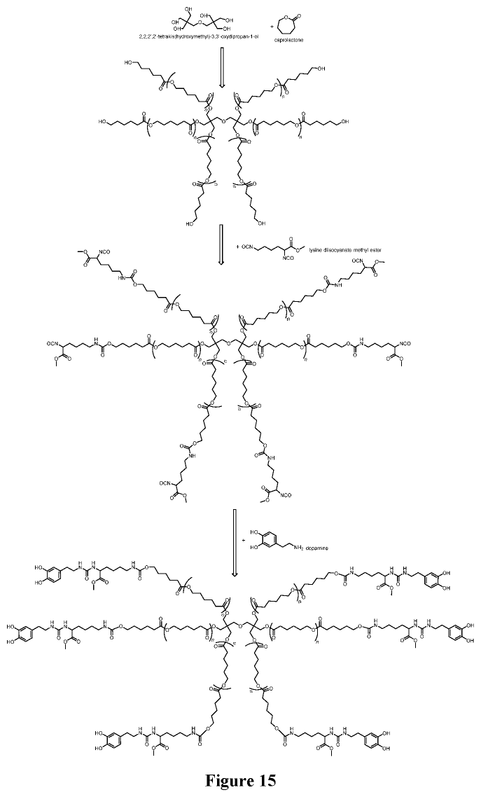

FIG. 15 shows a generalized reaction scheme for the synthesis of hexafunctional PCL adhesive in which the terminal hydroxyl groups of a PCL-Hexol reacts with one of two isocyanate groups of the diisocyanate molecule resulting in a PCL-Triisocyanate molecule. The free isocyanates then react with a reactive moiety (here an amine) present on the alkyl chain of the 4-alkylbenzene-1,2-diol derivative (here dopamine) to give Adhesive 2.

### Example 3: Difunctional Poly(Propylene Glycol) (PPG) Adhesive (Adhesive 3)

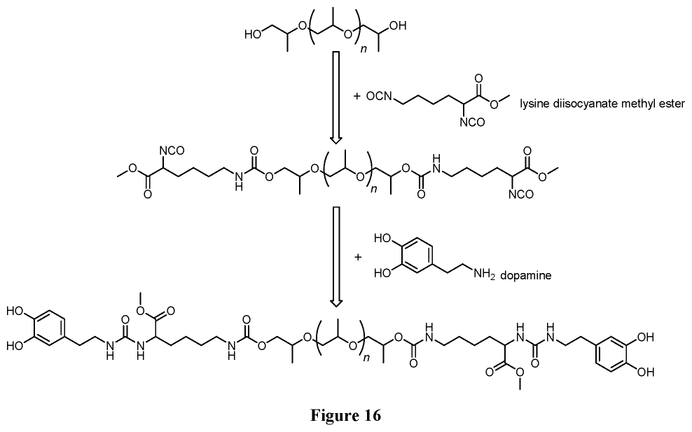

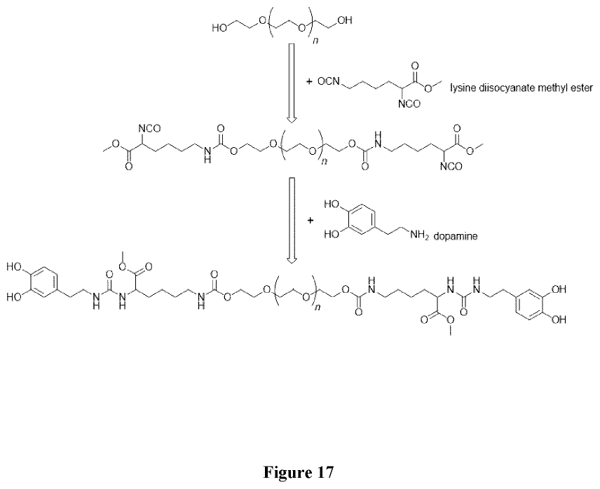

FIG. 16 shows a generalized reaction scheme for the synthesis of difunctional PPG adhesive in which the terminal hydroxyl groups of a PPG-Diol reacts with one of two isocyanate groups of the diisocyanate molecule resulting in a PPG-Diisocyanate molecule. The free isocyanates then react with a reactive moiety (here an amine) present on the alkyl chain of the 4-alkylbenzene-1,2-diol derivative (here dopamine).

### Example 3a: Synthesis of Adhesive 3a as an Example of Difunctional PPG Adhesive

### a) Activation of the Dopamine Derivative (Dopa):

Dopamine hydrochloride was activated as previously described. One molar equivalent of dopamine hydrochloride was dissolved into a minimum of anhydrous DMAc at room temperature. An equivalent amount of triethylamine was added to complex the hydrochloride salt from dopamine. The mixture was stirred for 30 minutes, yielding a cloudy solution. Triethylamine hydrochloride was separated from the activated dopamine by centrifuging for 10 minutes at 5000 rpm and −10° C. The activated dopamine was decanted from the solid triethylamine pellet and used immediately.

### b) Synthesis of Isocyanate Terminated PPG Oligomers (Prepolymer 3a):

21.69 g of dialcohol terminated poly(propylene glycol) with an average molecular weight of 1000 g/mol (n=17) was added to a dry 500 mL 2-neck flask and degassed under vacuum at 60° C. for 12 h. The flask was purged with argon and 100 ml anhydrous DMAc was added through a septum with stirring under argon protection. Once the reaction mixture was completely clear, it was cooled to room temperature. 9.66 g of LDI (5% molar excess to amine groups) was added to the reaction mixture with a syringe through a silicone rubber septum. The reaction mixture was then ramped to 60° C. over 4 hours, followed by a second ramp to 75° C. over an additional 4 hours. Finally, the reaction was stirred at 75° C. for 2 hours, then cooled to room temperature to give prepolymer 3a.

### c) Synthesis of PPG Based Oligomeric Dopamine Adhesive (Adhesive 3a)

Prepolymer 3a was functionalized by adding 9.07 g activated dopamine in 100 mL of DMAc to the reaction mixture. An ice bath was used to dissipate heat from this rapid and exothermic step. The reaction was stirred for an additional 2 hours to ensure complete reaction, then the mixture was slowly poured into a 1:1 mixture of hexanes and diethyl ether (500 mL) at room temperature. The mixture was stirred vigorously for 1 hour, then stirring was ceased and the reaction mixture was cooled in the fridge. The liquid phase was decanted off, and Adhesive 3a was recovered from the walls of the beaker. It was re-dissolved in acetone (250 mL) and precipitated by pouring into hexanes a second time (500 mL) to remove any residual reaction by-products.

### Example 4

### Example 4a: Synthesis of Adhesive 4a as an Example of a Trifunctional PEG Adhesive

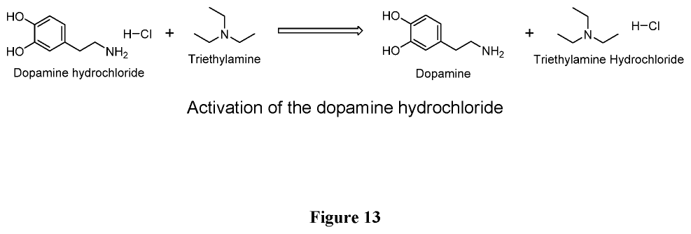

a) Preparation of Dopamine Derivative to React with Oligomer
The activation of dopamine hydrochloride from triethylamine resulted in an activated dopamine constituent. Dopamine hydrochloride was dissolved in anhydrous dimethylacetamide (DMAc) at a concentration of 0.5M for 10 minutes at room temperature. Triethylamine was added to the solution at a stoichiometric molar ratio of 1:0.95 to form triethylamine hydrochloride salt as it precipitated out from DMAc. The solution was centrifuged at 3420 rpm for 30 minutes at −10° C. to ensure the salt had formed a solid pellet, and the supernatant was used to cap the end groups of the oligomer in the examples below. FIG. 13 shows the activation of dopamine hydrochloride.

### b) Prepolymer Synthesis of Isocyanate Terminated Polyethylene Glycol Triol (PEG) Oligomers:

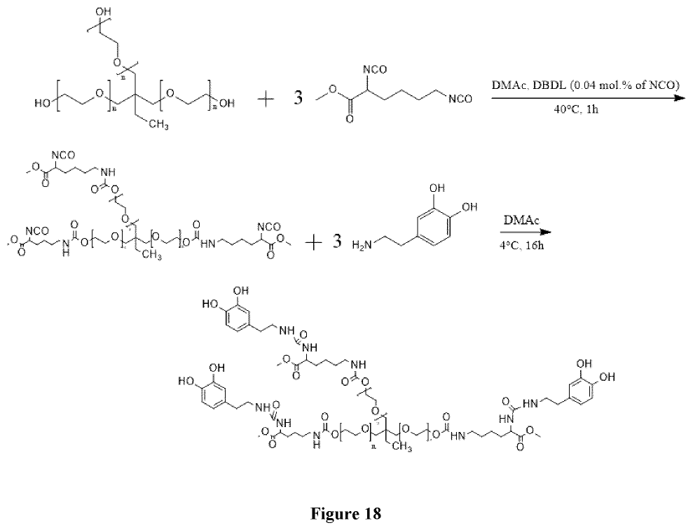

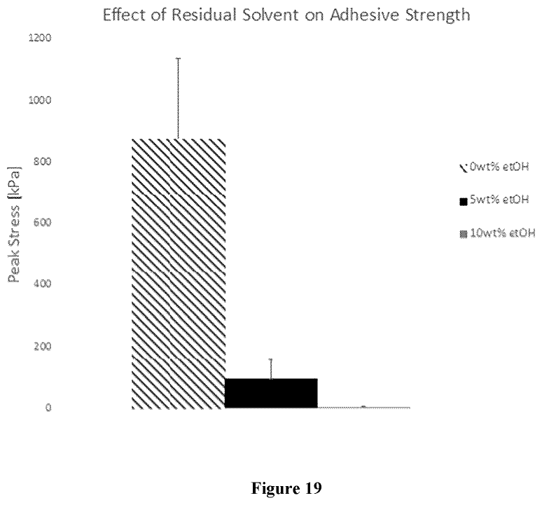

7.38 mmol of polyethylene glycol triol with an average molecular number of 1014 g/mol was added to a 50 mL two neck flask and degassed under vacuum at 60° C. for 12 hours. The flask was purged with argon and 30 mL of anhydrous DMAc was added through the rubber septum with constant stirring under argon. Once the triol was completely dissolved, it was cooled to room temperature and transferred into the glovebox along with DMAc, Methyl ester L-Lysine Diisocyanate (LDI) and Dibutyltin Dilaurate (DBDL). To start, 23.2 mmol of LDI (10% molar excess to the PEG hydroxyl groups) was added into a round bottom flask with 30 mL of DMAc and lastly 0.4 mol. % of DBTDL. The round bottom flask was added into an oil bath that was heated and mixed for 15 minutes. PEG was withdrawn with a 30 mL syringe added dropwise into the round bottom flask for a total duration of 9 minutes and remained stirring for an hour. After the hour, the round bottom flask was transferred from the glovebox into the fumehood which resulted into prepolymer 4a. This oligomer was then reacted with the activated dopamine to generate the structure in FIG. 18.

### c) Synthesis of Dopamine Terminated Polyethylene Glycol (PEG) Oligomer Diol Oligomers:

24.5 mmol of activated dopamine derivative (prepared according to Example 4a) was added into prepolymer 4a and allowed to mix overnight to ensure all functionalities of isocyanates have reacted. Upon completion of the reaction, the final product was filtered using a Buchner Funnel to remove remaining triethylamine hydrochloride prior to precipitation. The oligomer was added dropwise into 2 L of ethyl ether stirred at 800 rpm, where the oligomer was precipitated from DMAc. The solution was stirred overnight at room temperature or until the solution turned transparent to ensure all the oligomer has precipitated. The supernatant was decanted and approximately 25 mL of ethanol was added to dissolve the product, where 1 L of ethyl ether was added to re-precipitate the oligomer. This process was repeated three times to ensure the catalyst and DMAc were completely removed. On the final re-solubilization of the oligomer, ethyl ether was added into amber vials, where the product was added into these vials. Once the product had precipitated and the solvent was decanted, the final product was placed in a vacuum oven for 5 days to further remove any residual solvent.

### Example 5: Cytotoxicity Testing

Cytotoxicity Assays: In vitro cytotoxicity of adhesive formulations was assessed by direct contact with cells, according to ISO 10993-5 [35], which describes test methods to evaluate in vitro cytotoxicity of medical devices and, therefore, determine the in vitro biological response of mammalian cells using appropriate parameters. DNA mass quantification and Water-soluble tetrazolium (WST-1) assays were used to evaluate the toxicity of the formulations to a cell line. Negative and positive controls consisted of growth media with untreated cells and 5% Dimethyl sulfoxide (DMSO), respectively.

Cell Culture and Sample Preparation: A10 smooth muscle cell line (ATCC, CRL-1476™) were cultured in Dulbecco's modified Eagle's medium (DMEM), supplemented with 100 U mL⁻¹ penicillin/100 μg of streptomycin and 10% fetal bovine serum in a humidified atmosphere at 37° C. with 5% C02. The medium was changed every other day, allowing the cells to grow and reach confluency. When the cells reached 80-90% confluence, they were passaged following treatment with 0.25% trypsin-EDTA solution. This procedure was repeated until the cells entered their standard cellular cycle [36, 37]. Samples of adhesives were pre-conditioned in an incubator with DMEM for 24 h. Then, cells were seeded onto the samples, to which 200 μL of A10 suspension (approximately 25,000 cells/mL) was added. The seeded specimens were confined in the wells of a 96-well plate for 24 h, 72 h and 7 days.

DNA mass quantification: At each time-point, cells were lysed with ice-cold lysis buffer (0.05% Triton X-100, 50 mM EDTA in PBS) for 1 h and tested for DNA assay as previously described [37-39]. First, 0.1% Hoechst Dye 33258 was diluted in a Trizma base/NaCl/EDTA buffer (0.10 M Trizma base, 0.2 M NaCl and 0.001 M EDTA in ddH2O, pH 7.4) and 100 μL was added to a 96-well plate (Microfluor 2 Black, VWR). Then, 10 μL of cell lysate was added to the dye and read against a calf thymus DNA standard in a fluorescence microplate reader (BioTek Cytation 3, Biotek, Winooski, Vt., USA), with the excitation wavelength set at 360 nm and the emission wavelength observed at 460 nm [36].

WST-1 assay: For the WST assay, after removing media and rinsing with 1× with PBS, 200 μL of DMEM was added to each well. Next, 20 μL of the WST reagent was added to each well and the microplate will be incubated at 37° C. with 5% C02 for 1 h. The absorbance was read using a multi-mode reader (BioTek Cytation 3, Biotek, Winooski, Vt., USA) at 450 nm, with a reference wavelength of 650 nm [36]. Cell viability (%) was obtained using the equation:

$$Cell viability ( \% ) = [ 1 - \{ \frac{At - Ab}{At - Ac} \} ] \times 100. \qquad [ 40 ]$$

Statistical analysis: Data was analyzed using the software PASW Statistics 21.0 (SPSS Inc, Chicago, Ill., USA). Two-Way analysis of variance (ANOVA) and Tukey's multiple comparison post-hoc tests will be used to determine a statistical significance between the groups for the DNA mass quantification and WST-1 assays. Homogeneity of variance and normality were verified with Levene's and Shapiro-Wilk tests, respectively. The confidence interval for all the tests will be set at 95%.

TABLE 1
DNA assay results for A10 cells exposed to select adhesives after 24 h, 72 h and
7 days incubation at 37° C. with 5% CO₂. Cell viability results are relative to values for
DMEM negative control at each time point. 5% DMSO was used as a positive control.
Results are expressed as Mean ± Standard Deviation, n = 4.

| | Relative DNA mass (%) | | |
|---|---|---|---|
| Group | 24 h | 72 h | 7 d |
| DMEM (Negative control) | 100.0 ± 9.2 | 100.0 ± 12.1 | 100.0 ± 21.1 |
| 5% DMSO (Positive control) | 15.3 ± 5.3 | 1.3 ± 1.9 | 1.4 ± 1.6 |
| Adhesive 1b | 102.6 ± 16.2 | 87.0 ± 6.0 | 89.2 ± 7.2 |
| Adhesive 2 | 128.8 ± 13.0 | 109.9 ± 28.2 | 71.9 ± 5.5 |
| Adhesive 3a | 52.0 ± 2.3 | 44.4 ± 9.6 | 62.7 ± 22.7 |

TABLE 2
WST-1 cell viability assay results for A10 cells exposed to select adhesives after 24 h, 72 h
and 7 days incubation at 37° C. with 5% CO₂. Cell viability results are relative to values for
DMEM negative control at each time point. 5% DMSO was used as a positive control.
Results are expressed as Mean ± Standard Deviation, n = 4.

| | Relative Cell viability (%) | | |
|---|---|---|---|
| Group | 24 h | 72 h | 7 d |
| DMEM (Negative control) | 100.0 ± 6.5 | 100.0 ± 5.8 | 100.0 ± 12.3 |
| 5% DMSO (Positive control) | 18.4 ± 0.9 | 4.4 ± 1.1 | 1.0 ± 0.5 |
| Adhesive 1b | 77.1 ± 2.4 | 79.7 ± 10.0 | 74.0 ± 10.0 |
| Adhesive 2 | 83.5 ± 2.4 | 70.6 ± 8.0 | 74.5 ± 24.3 |
| Adhesive 3a | 103.7 ± 8.2 | 66.4 ± 8.1 | 46.4 ± 12.0 |

Tables 1 and 2 show that the PPG adhesive systems exhibited cytocompatibility (≥70%) after at least 24 hrs of contact with the A10 cell lines, indicating their suitability for use in medical device applications.

### Example 6: Determination of the Thermal Properties of a Bioadhesive

Melting and glass transition data was obtained by modulated differential scanning calorimetry (mDSC). Calorimetry studies were performed on a DSC Q200 (TA Instruments, Newcastle, Del., U.S.A.) equipped with a RCS90 refrigerated cooling system. Each sample (4-8 mg) was hermetically sealed into an aluminium DSC pan, and subsequently introduced into the DSC sample chamber continuously which was purged with a dry nitrogen flow of 60 mL/min. The sample was equilibrated at 100° C. for 2 minutes to erase thermal history. A modulation frequency with an amplitude of 1.0° C./minute was initiated, and the sample was then cooled at a rate of 10.0° C./min down to −80° C. where it was held isothermally for 2 min. Finally, the sample was heated from −80° C. to 100° C. at a rate of 10.0° C./min and held isothermally for an additional 2 minutes. Thermal properties were calculated from the thermograms with TA Universal Analysis 2000 software.

TABLE 3
Glass transition temperature (Tg) of adhesives and mixtures as determined by DSC from
the midpoint of the Tg transition curve on the heating cycle. Samples were run in hermetically
sealed aluminium DSC pans with 3-6 mg of sample per run. Tg values determined by the TA
Universal Analysis 2000 software from samples run at 10° C./min with a continuous modulation
at an amplitude of 1.0° C./minute. Comparison between heat flow, reversable heat flow, and
non-reversable heat flow thermograms were used to isolate the glass transition events based
on their second order phase transition behaviour.

| | Glass Transition Temperature- |
|---|---|
| Adhesive | Tg (° C.) |
| Adhesive 1a | −1.2 ± 1.0 |
| Adhesive 1b | −21.5 ± 1.8 |
| Adhesive 2 | 14.2 ± 3.0 |
| Adhesive 3a | −6.8 ± 3.5 |
| Mixture (wt/wt %) 60% Adhesive 2a + 40% Adhesive 1b | 0.7 ± 2.3 |
| Mixture (wt/wt %) 70 Adhesive 2a + 30% Adhesive 1b | 6.6 ± 2.0 |
| Mixture (wt/wt %) 80% Adhesive 2a + 20% Adhesive 1b | 11.9 ± 0.6 |
| Mixture (wt/wt %) 90% Adhesive 2a + 10% Adhesive 1b | 18.0 ± 0.3 |

Differential scanning calorimetry was used to determine the transition temperatures of the bioadhesives described herein. None of the adhesives displayed sharp or well-defined melting points, instead they all possessed well defined and narrow glass transition temperatures. These indicated the absence of crystalline domains and the inherent nature of their amorphous state, respectively. Higher glass transition temperatures are typical of glassy materials, whilst materials with lower glass transition temperatures remain amorphous for longer/until lower temperatures. Variations in the glass transition temperatures were observed related to the inherent glass transition temperatures of the oligomeric backbone chemistry and were also related to oligomer length and catechol functionalization degree. Mixtures of adhesives generally displayed only a single glass transition temperature between the range of the two mixtures, and the transition temperature was more heavily weighted towards the major weight constituent of the mixture.

### Example 7: Testing Adhesion Strength

Adhesion strength is a major design criterion for any adhesive. Here we define adhesion strength in shear, in either compression or tension depending on the sample type. Adhesion strength values were obtained from the maximum stress (converted to Pa from the applied force and cross-sectional area in contact between the two substrates tested). Adhesion was tested using a variety of temperatures, substrates, and formulations with and without induced crosslinking.

In general, adhesives with higher crosslinking density (higher content of catechol derivatives per weight), higher melting temperatures and/or higher Tg values were less elastic, with little or no plastic deformation and a catastrophic failure mechanism. Without wishing to be bound by a theory, this is typical of materials with reduced elasticity and reflective of elevated crosslinking and/or being tested in a rigid or glassy state where there is limited plastic deformation of the adhesive oligomers, thus limiting rearrangement and elastic deformation or strain hardening. The less rigid adhesives generally failed by a ductile failure mode following a degree of plastic deformation.

### Example 7a: The Effect of Crosslinkers and Substrates

To determine the effect of different crosslinkers on adhesion strength, several oxidants and complexing agents were mixed into adhesives at different weight ratios and applied to samples and allowed to cure. Adhesion strength was measured using a compressive shear test.

Crosslinking agents in this test included the inorganic oxidants Na₃VO₄ and tetrabutylammonium (meta)periodate (TBAP), an inorganic base (Na₂CO₃), an organic complexing salt (iron (III) citrate—FeCit), and two inorganic complexing salts (FeCl₃ and FePO₄). Briefly, crosslinkers were hand mixed into different adhesives on glass slides with the aid of heat and immediately applied onto aluminum, ceramic or glass substrates using a contact area of 90 mm². Samples were loaded in shear on an Instron 4301 universal testing machine under modified ASTM D905-08 conditions.

TABLE 4a
Adhesion properties of bioadhesives using varying crosslinking agents and concentrations.
Testing was conducted on an Instron 4301 universal testing machine with a 1000 N load cell
and an applied strain rate of 2 mm/min at ambient room temperature on standard sized samples
consisting of two pieces of dry aluminium or ceramic or glass adherends with approximately
0.20 g of adhesive between surfaces. Contact surface areas were kept nearly constant
(~90 mm²) for all samples in the set. Results are expressed as Mean ± standard deviation,
n = 4. Glass is defined as commercially available plain glass slides. Ceramic samples are
composed of calcium polyphosphate tablets.

| Adhesive, Crosslinking agent, Substrate | Adhesive Strength (MPa) |
|---|---|
| Adhesive 1a + 5% wt/wt Na₃VO₄ (Aluminium) | 2.7 ± 0.9 |
| Adhesive 1a + 2.5% wt/wt Na₃VO₄ (Aluminium) | 6.2 ± 1.9 |
| Adhesive 1a + 5% wt/wt Na₂CO₃ (Aluminium) | 3.0 ± 0.9 |
| Adhesive 1a + 2.5% wt/wt Na₂CO₃ (Aluminium) | 5.8 ± 2.8 |
| Adhesive 1a + 5% wt/wt Na₃VO₄ (Ceramic) | 1.2 ± 0.6 |
| Adhesive 1a + 5% wt/wt Na₂CO₃ (Glass) | 1.4 ± 0.5 |
| Adhesive 1a + 5% wt/wt TBAP (Aluminium) | 8.2 ± 2.7 |
| Adhesive 1a + 2.5% wt/wt TBAP (Aluminium) | 8.4 ± 2.2 |
| Adhesive 1a + 5% wt/wt FeCl₃ (Aluminium) | 1.7 ± 0.5 |
| Adhesive 1a + 5% wt/wt FePO₄ (Aluminium) | 1.9 ± 0.9 |
| Adhesive 1a + 5% wt/wt FeCit (Aluminium) | 2.6 ± 0.7 |
| Adhesive 1b + 5% wt/wt Na₂CO₃ (Aluminium) | 0.9 ± 0.5 |
| Adhesive 1b + 2.5% wt/wt Na₂CO₃ (Aluminium) | 1.6 ± 0.9 |
| Adhesive 1b + 5% wt/wt Na₃VO₄ (Aluminium) | 1.2 ± 0.6 |
| Adhesive 1b + 2.5% wt/wt Na₃VO₄ (Aluminium) | 0.8 ± 0.5 |
| Adhesive 1b + 5% wt/wt Na₃VO₄ (Ceramic) | 0.3 ± 0.2 |
| Adhesive 1b + 5% wt/wt TBAP (Aluminium) | 1.7 ± 0.5 |
| Adhesive 1b + 2.5% wt/wt TBAP (Aluminium) | 0.8 ± 0.3 |
| Adhesive 1b + 5% wt/wt FePO₄ (Aluminium) | 0.6 ± 0.3 |
| Adhesive 1a + Adhesive 1b + 5% wt/wt Na₂CO₃ | 1.6 ± 0.5 |
| (Aluminium) | |
| Adhesive 1a + Adhesive 1b + 5% wt/wt Na₂CO₃ (Glass) | 1.9 ± 0.2 |

### Example 7b: The Effect of Temperature on Adhesive Strength

Without crosslinking, adhesives are vulnerable to the temperature they are tested at, as with all thermoplastic/hotmelt adhesives. Thus, samples were tested at temperatures above and below their glass transition temperatures. Glass transition temperatures were determined using dynamic scanning calorimetry (DS) and are reported in Table 4c. No oxidants were used in these experiments to induce crosslinking. Therefore, the results presented reflect the natural cohesive strengths of the reported dry adhesive formulations.

TABLE 4b
Mechanical properties of bioadhesives tested approximately 20° C. above and 20° C. below the
midpoint of their glass transition temperature (as determined by DSC) without crosslinking
agents on rectangular aluminium stubs under modified ASTM D905-08 conditions. Adhesive
mixtures were prepared by cryogenic milling. Approximately 60 mg of the adhesive was applied
evenly over the 2 cm² surface area. Testing was conducted after samples stabilized at the required
testing temperatures. Data obtained by compression lap shear strength testing using Instron 4301
universal testing machine, with a 1000 N load cell and an applied strain rate of 5.0 mm/min.
Results are expressed as Mean ± standard deviation, n = 3.

| | | Test | | |
|---|---|---|---|---|
| | Tg | Temp | Stress (MPa) | |
| Adhesive | (° C. on heat) | [° C.] | Above Tg | Below Tg |
| Adhesive 3a | −6.8 ± 3.5 | −10, 37 | 0.33 ± 0.17 | 3.71 ± 0.31 |
| Adhesive 2 | 14.2 ± 3.0 | 4, 37 | 1.07 ± 0.47 | 1.28 ± 0.62 |
| Adhesive 1b | −21.5 ± 1.8 | −80, 4 | 0.16 ± 0.08 | 3.84 ± 1.13 |
| Mixture (wt/wt %)-90 Adhesive 2/ | 18.0 ± 0.3 | 4, 37 | 1.36 ± 0.31 | 1.54 ± 0.64 |
| 10 Adhesive 1b | | | | |
| Mixture (wt/wt %)-80 Adhesive 2/ | 11.9 ± 0.6 | 4, 37 | 0.92 ± 0.42 | 1.52 ± 0.91 |
| 20 Adhesive 1b | | | | |
| Mixture (wt/wt %)-70 Adhesive 2/ | 6.6 ± 2.0 | −20, 37 | 0.87 ± 0.48 | 1.59 ± 0.08 |
| 30 Adhesive 1b | | | | |
| Mixture (wt/wt %)-60 Adhesive 2/ | 0.7 ± 2.3 | −20, 37 | 0.48 ± 0.38 | 1.19 ± 0.51 |
| 40 Adhesive 1b | | | | |

In formulations with glass transitions far below 37° C., the adhesive strengths are considerably reduced at physiological temperatures. However, when doped with another adhesive with a Tg closer to 37° C., the glass transitions will shift towards higher temperatures, allowing these materials to be functional at physiological temperatures, thereby increasing their utility. In addition or alternatively, cross-linking agents may be used to improve adhesive strength.

### Example 8: Testing Effect of Residual Solvent on Adhesive Performance

Residual solvent is a major challenge for bioadhesives. Synthesis in the presence of a solvent requires rigorous drying protocols to fully remove solvent, as solvents are often cytotoxic, and could accelerate in water ingress.

To test the effect of residual solvent on mechanical properties of uncrosslinked adhesive increasing amounts of solvent were incorporated into adhesive and mechanical properties were measured using a modified lap shear test. This experiment was designed to mimic both incomplete drying of the adhesive after synthesis, and what occurs when solvents are utilized for adhesive spreading and/or adhesive application.

To incorporate increasing amounts of solvent into adhesives, the process started with dry Adhesive 2. Using cryogenic milling 0, 5, 10 and 50 wt % anhydrous ethanol were incorporated into aliquots of adhesive. Briefly, ethanol was added dropwise to a specific mass percent of the adhesive. Adhesive/solvent mixtures were added to a large 50 mL steel milling cannister containing 4 ball-bearings. Adhesive/ethanol mixtures were homogenized using cryogenic milling on a Retsch cryomill at −196° C. Mixtures were milled for 3 cycles at 30 Hz with −5 minutes of cooling between cycles. Adhesive-ethanol mixtures were transferred to scintillation vials and stored at −20° C. before use.

Aluminum stubs were prepared and cleaned with acetone. The surface of each stub was smooth, and a 2×1 cm area was indicated to ensure constant overlap area. ˜60 mg of each adhesive-ethanol mixture was spread onto the stubs and an overlapping stub was applied by hand and pressed together for ˜10 s. 0 and 5 wt % ethanol adhesives required some heat for spreading. Overlap area was held constant at 2 cm². Values could not be obtained for 50 wt % ethanol adhesive, as the cohesive strength was insufficient to hold the aluminum stubs together. Even at 10 wt % ethanol, the adhesive exhibited insufficient strength; the force of gravity resulted in deformation. 4 repeats of 0, 5, and 10 wt % ethanol adhesives were prepared and kept at room temperature for ˜1 hr before testing.

Samples were tested on a Universal Testing Machine (Instron) under compression, using a modified lap shear test, at a strain rate of 5 mm/min. Stress and displacement data were recorded.

Increasing amounts of residual ethanol affected the cohesive strength of Adhesive 2. FIG. 19 shows adhesive strength with increasing residual solvent. 5 wt % ethanol reduced adhesive strength by 9×. Data was generated in compression using a modified lap shear test at a strain rate of 5 mm/min on aluminum stubs with an overlap area of 2 cm². Samples were tested at room temperature. Error bars indicate standard deviation (n=4). Doubling solvent to 10 wt % further reduced adhesive strength by ˜30×.

Clearly, increasing amounts of residual solvent decreases the cohesive strength of adhesives.
P. Spencer, Q. Ye, J. Park, E. M. Topp, A. Misra, O. Marangos, Y. Wang, B. S. Bohaty, V. Singh, F. Sene, Adhesive/dentin interface: the weak link in the composite restoration, Annals of biomedical engineering 38(6) (2010) 1989-2003.
B. Petersen, A. Barkun, S. Carpenter, P. Chotiprasidhi, R. Chuttani, W. Silverman, N. Hussain, J. Liu, G. Taitelbaum, G. G. Ginsberg, Tissue adhesives and fibrin glues: November 2003, Gastrointestinal endoscopy 60(3) (2004) 327-333.
W. D. Spotnitz, Fibrin sealant: the only approved hemostat, sealant, and adhesive—a laboratory and clinical perspective, ISRN surgery 2014 (2014).
R. Chivers, R. Wolowacz, The strength of adhesive-bonded tissue joints, International journal of adhesion and adhesives 17(2) (1997) 127-132.
I. Peripheral, Toxicity of Alkyl 2-Cyanoacrylates.
D. Lu, H. Wang, T.e. Li, Y. Li, X. Wang, P. Niu, H. Guo, S. Sun, X. Wang, X. Guan, Versatile Surgical Adhesive and Hemostatic Materials: Synthesis, Properties, and Application of Thermoresponsive Polypeptides, Chemistry of Materials 29(13) (2017) 5493-5503.
D. F. Farrar, Bone adhesives for trauma surgery: A review of challenges and developments, International Journal of Adhesion and Adhesives 33 (2012) 89-97.
L. Sanders, J. Nagatomi, Clinical applications of surgical adhesives and sealants, Critical Reviews™ in Biomedical Engineering 42(3-4) (2014).
R. Bitton, E. Josef, I. Shimshelashvili, K. Shapira, D. Seliktar, H. Bianco-Peled, Phloroglucinol-based biomimetic adhesives for medical applications, Acta biomaterialia 5(5) (2009) 1582-1587.
M. Yu, J. Hwang, T. J. Deming, Role of L-3, 4-dihydroxyphenylalanine in mussel adhesive proteins, Journal of the American Chemical Society 121(24) (1999) 5825-5826.
H. Lee, B. P. Lee, P. B. Messersmith, A reversible wet/dry adhesive inspired by mussels and geckos, Nature 448(7151) (2007) 338.
B. P. Lee, P. B. Messersmith, J. N. Israelachvili, J. H. Waite, Mussel-inspired adhesives and coatings, Annual review of materials research 41 (2011) 99-132.
J. H. WAITE, Adhesion in byssally attached bivalves, Biological Reviews 58(2) (1983) 209-231.
H. G. Silverman, F. F. Roberto, Understanding marine mussel adhesion, Marine biotechnology 9(6) (2007) 661-681.
B. P. Lee, J. L. Dalsin, J. L. Murphy, L. Vollenweider, A. N. Lyman, F. Xu, J. White, W. D. Lew, M. Brodie, Adhesive compounds and methods use for hernia repair, Google Patents, 2016.
B. P. Lee, L. Vollenweider, J. L. Murphy, F. Xu, J. L. Dalsin, J. Virosco, W. Lew, J. White, Bioadhesive constructs, Google Patents, 2013.
P. B. Messersmith, J. L. Dalsin, B. P. Lee, S. A. Burke, DOPA-functionalized, branched, poly (aklylene oxide) adhesives, Google Patents, 2014.
J. L. Dalsin, B. P. Lee, L. Vollenweider, S. Silvary, J. L. Murphy, F. Xu, A. Spitz, A. Lyman, Multi-armed catechol compound blends, Google Patents, 2014.
B. P. Lee, S. Silvary, J. L. Murphy, Multibranched bioadhesive compounds and synthetic methods therefor, Google Patents, 2017.
P. B. Messersmith, L. He, D. E. Fullenkamp, pH responsive self-healing hydrogels formed by boronate-catechol complexation, Google Patents, 2017.
M. Szycher, Szycher's dictionary of biomaterials and medical devices, Routledge2019.
E. J. Beckman, M. Buckley, S. Agarwal, J. Zhang, Medical adhesive and methods of tissue adhesion, Google Patents, 2007.
M. Giannini, C. A. G. Arrais, P. M. Vermelho, R. Reis, L. Santos, E. R. Leite, Effects of the solvent evaporation technique on the degree of conversion of one-bottle adhesive systems, Operative dentistry 33(2) (2008) 149-154.
I. V. Luque-Martinez, J. Perdigso, M. A. Munoz, A. Sezinando, A. Reis, A. D. Loguercio, Effects of solvent evaporation time on immediate adhesive properties of universal adhesives to dentin, Dental Materials 30(10) (2014) 1126-1135.
M. Suzuki, S. Tsuge, T. Takeuchi, Gas chromatographic estimation of occluded solvents in adhesive tape by periodic introduction method, Analytical Chemistry 42(14) (1970) 1705-1708.
F. Scognamiglio, A. Travan, I. Rustighi, P. Tarchi, S. Palmisano, E. Marsich, M. Borgogna, I. Donati, N. de Manzini, S. Paoletti, Adhesive and sealant interfaces for general surgery applications, Journal of Biomedical Materials Research Part B: Applied Biomaterials 104(3) (2016) 626-639.
A. Duarte, J. Coelho, J. Bordado, M. Cidade, M. Gil, Surgical adhesives: Systematic review of the main types and development forecast, Progress in Polymer Science 37(8) (2012) 1031-1050.
P. Kord Forooshani, B. P. Lee, Recent approaches in designing bioadhesive materials inspired by mussel adhesive protein, Journal of Polymer Science Part A: Polymer Chemistry 55(1) (2017) 9-33.
N. Annabi, A. Tamayol, S. R. Shin, A. M. Ghaemmaghami, N. A. Peppas, A. Khademhosseini, Surgical materials: Current challenges and nano-enabled solutions, Nano today 9(5) (2014) 574-589.
W. D. Spotnitz, S. Burks, Hemostats, sealants, and adhesives: components of the surgical toolbox, Transfusion 48(7) (2008) 1502-1516.
K. A. Vakalopoulos, F. Daams, Z. Wu, L. Timmermans, J. J. Jeekel, G.-J. Kleinrensink, A. van der Ham, J. F. Lange, Tissue adhesives in gastrointestinal anastomosis: a systematic review, Journal of Surgical Research 180(2) (2013) 290-300.
G. Odian, Principles of polymerization, John Wiley & Sons 2004.
C. M. Whyne, R. M. Pilliar, J. A. Fialkov, P. Santerre, E. Regev, Bone stabilization device and method of production, European Patent Office, 2015, p. EP2879600A4.
C. M. Whyne, R. M. Pilliar, J. A. Fialkov, P. Santerre, E. Regev, Bone stabilization device and method of production, in: W.I.P. Organization (Ed.) WO2014019083A1 2015.
Iso, Biological evaluation of medical devices-Part 5: Tests for in vitro cytotoxicity, International Organization for Standardization Geneve, Switzerland, 2009.
J. W. C. Cheung, E. E. Rose, J. P. Santerre, Perfused culture of gingival fibroblasts in a degradable/polar/hydrophobic/ionic polyurethane (D-PHI) scaffold leads to enhanced proliferation and metabolic activity, Acta biomaterialia 9(6) (2013) 6867-6875.
S. Sharifpoor, C. A. Simmons, R. S. Labow, J. P. Santerre, A study of vascular smooth muscle cell function under cyclic mechanical loading in a polyurethane scaffold with optimized porosity, Acta biomaterialia 6(11) (2010) 4218-4228.
L. A. Matheson, G. N. Maksym, J. P. Santerre, R. S. Labow, Differential effects of uniaxial and biaxial strain on U937 macrophage-like cell morphology: Influence of extracellular matrix type proteins, Journal of Biomedical Materials Research Part A 81(4) (2007) 971-981.
L. A. Matheson, J. P. Santerre, R. S. Labow, Changes in macrophage function and morphology due to biomedical polyurethane surfaces undergoing biodegradation, Journal of cellular physiology 199(1) (2004) 8-19.
P. Makvandi, M. Ghaemy, A. A. Ghadiri, M. Mohseni, Photocurable, Antimicrobial Quaternary Ammonium-modified Nanosilica, Journal of dental research (2015) 0022034515599973.

1. An adhesive comprising:
a compound of structure 1 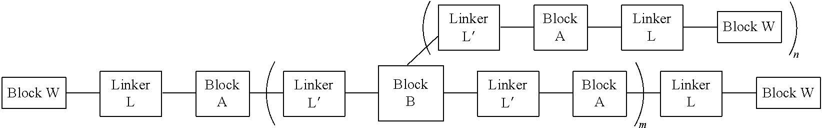
Structure I
wherein
the compound has a Tg lower than 25° C.;
wherein the compound is water soluble;
B is a branched or unbranched oligomer derived from a polysaccharide, polyamide, polyether, or polyalkylene glycol, with a MW<5,000 g/mol;
Linker L is an urethane, urea bond, or amide bond;
Linker L′ is an urethane or urea bond;
A is a chain extender of Mw≤3000 g/mol comprising substituted or unsubstituted alkyl, cycloalkyl and/or aromatic groups;
W is a terminal adhesive benzene-1,2-diol derivative or a terminal adhesive benzene-1,2,3-triol derivative;
m is 0 or 1; and
n is 0, 1, 2, 3 or 4;
or
a cross-linked polymer produced by cross-linking compounds of structure 1.

2. (canceled)

3. The adhesive of claim 1 wherein m is 1 and n is 0 and the adhesive comprises a compound of structure 3 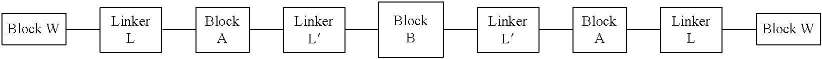
Structure 3
or
a cross-linked polymer produced by cross-linking compounds of structure 3.

4. The adhesive of claim 1 wherein m is 1 and n is 1 and the adhesive comprises a compound of structure 4 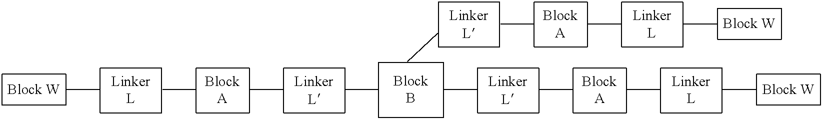
Structure 4
or
a cross-linked polymer produced by cross-linking compounds of structure 4.

5. The adhesive of claim 1 wherein m is 1 and n is 2 and the adhesive comprises a compound of structure 5 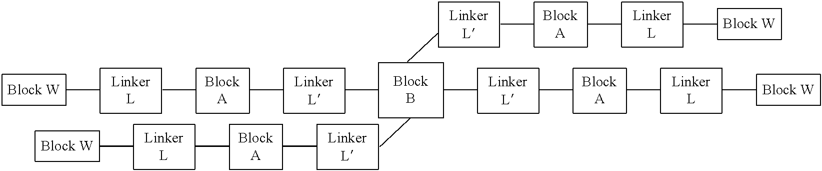
Structure 5
or
a cross-linked polymer produced by cross-linking compounds of structure 5.

6. The adhesive of claim 1 wherein m is 1 and n is 3 and the adhesive comprises a compound of structure 6 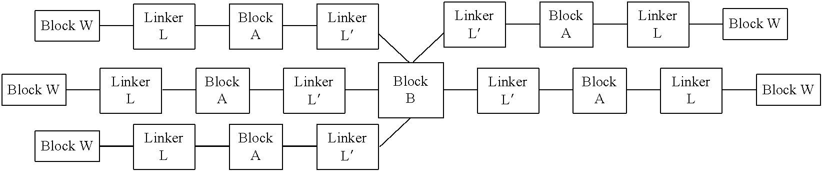
Structure 6
or
a cross-linked polymer produced by cross-linking compounds of structure 6.

7. The adhesive of claim 1 wherein m is 1 and n is 4 and the adhesive comprises a compound of structure 7 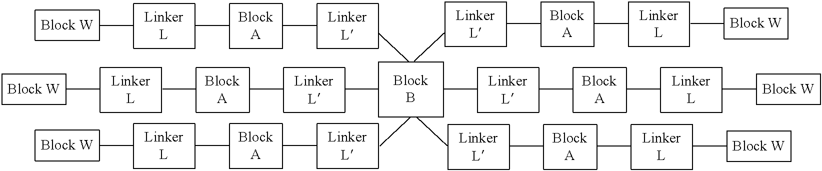
Structure 6
or
a cross-linked polymer produced by cross-linking compounds of structure 7.

8. The adhesive of claim 1 wherein the compound has a Tm equal to or greater than 37° C.

9. The adhesive of claim 1 wherein B has a Mw<3,000 g/mol, preferably between 500 and 1500 g/mol, and/or A has a MW<200 q/mol.

10. The adhesive of claim 1 comprising a compound of structure 1 and crosslinking agent or a curing agent in an amount between about 0 and 5%.

11. The adhesive of claim 1 wherein B is an oligomer derived from a polyether or a polyalkylene glycol, preferably polypropylene glycol or polyethylene glycol.

12. The adhesive of claim 1 wherein A is substituted with a C1-C6 carboxylate group.

13. (canceled)

14. The adhesive of claim 1 wherein W is a 4-alkylbenzene-1,2-diol derivative or a 5-alkylbenzene-1,2,3-triol derivative, optionally wherein W has a structure selected from: 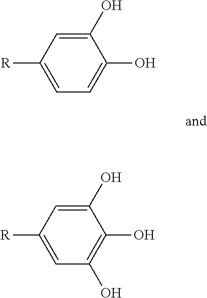
wherein R is present or absent and when present is a C1-C6 alkyl group or C1-C6 alkene optionally substituted with OH, NH.

15. (canceled)

16. The adhesive of claim 1 comprising a compound having m is 1 and n is 0 and another compound having m is 1 and n is 0, 1, 2, 3 or 4.

17. (canceled)

18. The adhesive of claim 1 consisting or consisting essentially of one or more compounds of structure 1 or a cross-linked polymer produced by cross-linking compounds of structure 1 or wherein one or more compounds of structure 1 or a cross-linked polymer produced by cross-linking compounds of structure 1 are the sole adhesive compounds in the adhesive.

19. (canceled)

20. The adhesive of claim 1 having a viscosity sufficient to be expelled out of a syringe at a temperature of at least 20° C.

21. The adhesive of claim 1 having an adhesive strength greater than 25 KPa under compression, tensile, or tensile lap shear testing.

22. (canceled)

23. The adhesive of claim 1 further comprising a biological agent, pharmaceutical agent or diagnostic agent covalently bound or complexed to the compound or entrapped within the adhesive.

24. (canceled)

25. The adhesive of claim 1, wherein the adhesive comprises 5-95% by weight compounds of structure 1 and 5-95% by weight of a biocompatible polymer or oligomer, optionally a biodegradable polyester.

26. (canceled)

27. A method of adhering a first surface and a second surface comprising:
applying the water soluble adhesive of claim 1 neat to at least a portion of the first surface; and
bringing at least a portion of the second surface into contact with the adhesive.

28. (canceled)

29. (canceled)

30. The method of claim 27 further comprising applying an external energy source to the adhesive and/or at least one of the first and second surface to enhance adhesion or application.

31. The adhesive of claim 1 wherein the compound is prepared by
reacting a first isocyanate group of a diisocyanate having a substituted or unsubstituted alkyl-derived chain extender, a substituted or unsubstituted cycloalkyl-derived chain extender, or a substituted or unsubstituted benzyl-derived chain extender between the two isocyanate groups
with terminal amino or alcohol groups of an oligomer having at least two terminal amino or alcohol groups and reacting a second isocyanate of the diisocyanate molecule with an amine group, alcohol group or carboxy group present on an alkyl chain of a 4-alkylbenzene-1,2-diol derivative or a 5-alkylbenzene-1,2,3-triol derivative.

32. (canceled)

33. (canceled)

34. (canceled)
